(19) United States
(12) Patent Application Publication
(10) Pub. No.: US 2009/0233357 A1
(43) Pub. Date: Sep. 17, 2009

(54) Targeted Delivery of Compounds Using Multimerization Technology

(76) Inventors: Abedelnasser Abulrob, Ottawa (CA); Jianbing Zhang, Ottawa (CA)

Correspondence Address: NATIONAL RESEARCH COUNCIL OF CANADA;1200 MONTREAL ROAD, BLDG M-58, ROOM EG12, OTTAWA, ONTARIO, K1A 0R6, CA

(21) Appl. No.: 11/992,656

(22) PCT Filed: Sep. 15, 2006
(86) PCT No.: PCT/CA2006/001528

§ 371 Date: Mar. 27, 2008

Related U.S. Application Data
(60) Provisional application No. 60/720,452, filed on Sep. 27, 2005.

(51) Int. Cl. C12N 5/06; C07K 16/18

(52) U.S. Cl. 435/375; 530/387.3

(57) ABSTRACT

There is disclosed herein subunits and multimers of subunits suitable for use in inducing the transport of one or more cargo substances into a cell and in some instances across a cell. The subunits may have a targeting domain such a antibody or antibody fragment, a multimerization domain, such as a verotoxin B-subunit mutant scaffold, and a cargo molecule such as a drug or imaging agent, which may be directly linked to the subunit or may be packaged in a liposome, nanoparticle, or the like. In some instances the targeting domain may have affinity for a blood-brain barrier antigen and may be capable of inducing cell mediated transcytosis to facilitate delivery of the cargo molecule across the blood-brain barrier. In some instances the targeting region may have affinity for a cancer antigen and may be capable of inducing cell-mediated endocytosis.

This application claims the benefit of priority from U.S. patent application Ser. No. 60/720,452 filed Sep. 27, 2005.

### FIELD OF THE INVENTION

The invention relates to the field of targeted delivery of compounds in biological systems.

### BACKGROUND TO THE INVENTION

It is desirable to have a means to deliver compounds or materials of interest to specific tissues within a living organism. Various approaches have been employed to achieve this goal, including the use of antibodies or antibody fragments specific for an epitope found on cell types of interest. (See for example WO2004/078097 and WO 2005/052158 and U.S. 20040161738.)

However, effective delivery can still prove to be challenging. For example, the cells may not bind the antibody with high enough affinity, or they may not take up the bound complex either within the cell or across the cell.

Approaches which allow for the multimerization of molecules with specific binding affinities are known. (See for example WO 2003/046560.) However, multimeric complexes can display very different physical and pharmacological properties than their monomeric counterparts. Thus, it was not clear if, or how, multimerization technology could be employed to increase biological uptake or transport, particularly in systems containing multiple cell types, as is observed in vivo.

For example, the brain is isolated from the rest of the body by a specialized endothelial tissue known as the blood-brain barrier (BBB). The endothelial cells of the BBB are connected by tight junctions and efficiently prevent many therapeutic compounds from entering the brain.

### BRIEF DESCRIPTION OF THE INVENTION

The invention relates to subunits and multimers of subunits suitable for use in inducing the transport of one or more cargo substances into a cell and in some instances across a cell.

### BRIEF DESCRIPTION OF FIGURES

FIG. 1. Depicts a schematic of the expression vector used to engineer pentameric construct of FC5 (P5) on a verotoxin subunit B scaffold.

FIG. 2. Depicts functional assessment of the example of pentameric blood brain barrier-targeting antibody FC5, P5

FIG. 3. Depicts binding of P5 to isolated vessels and transmigration across in vitro model of the blood-brain barrier (human brain endothelial cells)

FIG. 4. Depicts the accumulation of FC5 antibody in the head region after i.v. injection.

FIG. 5. Depicts optical tomography (A) and 3D reconstruction (B) of the head region of FC5- vs. NC11-injected animals.

FIG. 6. Depicts a comparison of brain targeting by FC5 and P5 using in vivo optical imaging

FIG. 7. Depicts detection of Cy5.5-FC5 in the sections of various organs by confocal microscopy.

FIG. 8. Depicts ex-vivo confocal laser microscopy results.

FIG. 9. Depicts co-localization of Cy5.5-P5 antibody with neuronal marker (NeuN) in A) brain frontal cortex and B) brain parietal cortex

FIG. 10. Is a schematic depiction of an experimental design of proteomics.

FIG. 11. Depicts proteomic analyses of trypsin-digested sdAbs and LCM-collected vessels/parenchyma after antibody injection in vivo.

FIG. 12. Depicts a summary of detected antibody-specific peptides in the brain parenchyma of saline-, FC5-, P5- or D38Z-injected animals.

FIG. 13. Depicts pharmacokinetics of an experiment involving monomeric FC5 and pentameric FC5 (P5) after intravenous injection via tail vein in mice.

FIG. 14. Depicts a schematic drawing of the approach used for site-specific PEGylation of FC5.

FIG. 15. In vivo imaging of animals injected with FC5 or diFC5-PEG

FIG. 16. Comparison of brain targeting by FC5 and diFC5-PEG using in vivo optical imaging

FIG. 17. Depicts a schematic drawing of liposome formulation targeted with FC5.

FIG. 18. Is a graphical representation of brain concentration of doxorubicin 24 h after i.v. injection

FIG. 19. Is a graphical depiction of brain concentration of doxorubicin 24 h after i.v. injection using a different approach

### DETAILED DESCRIPTION OF THE INVENTION

There are disclosed herein methods and compounds for providing multimeric complexes useful in causing binding to, internalization into and/or transport across cells of interest.

Multimeric complexes may be formed from subunits comprising a targeting region and a multimerization region. In some instances, the targeting region is an antibody or fragment thereof or has a polypeptide sequence obtainable from an antibody or fragment thereof. In some instances, the multimerization region is derived or derivable from a verotoxin B-subunit scaffold or mutant thereof. In some cases, substantially identical multimerization regions will be employed to form homomultimers. In some cases two or more complementary multimerization domains will be used to form heteromultimers.

Thus, in an embodiment of the invention there is provided a method of causing or enhancing binding to, internalization into or movement across a cell of interest of a cargo substance, said method comprising:
a) obtaining a subunit comprising a targeting region and a multimerization region, the targeting region having affinity for an epitope capable of causing receptor-mediated internalization (endocytosis) and/or receptor-mediated transcytosis;
b) functionally linking the cargo substance to the subunit, for example by conjugation or by encapsulating the cargo substance in a liposome or other suitable capsule having a binder on its surface;
c) allowing binding of one or more multimeric complexes on the cell type of interest.

It will be understood that a cargo substance may be any compound of interest, including a pharmaceutical, an imaging agent, a toxin, and/or a nanoparticle or liposome containing a material of interest (e.g. a pharmaceutical, toxin, imaging agent, siRNA etc.) or another suitable compound.

In some instances it may be desirable to link to the cargo substance one or more molecules having affinity for a target in the tissue of interest accessible after transmigration of the impermeable cellular layers, such as blood-brain barrier, intestinal epithelium, Sertoli cell layer in the testis, or other vascular endothelial layers to facilitate specific targeting of the cargo substance. In some instances it may be desirable to link to the cargo substance one or more molecules having affinity for a target or subcellular compartment in the cell type of interest after internalization into the cell.

Receptors that undergo receptor-mediated transcytosis across the blood-brain barrier (such as antigens recognized by FC5 and FC44) or other impermeable cellular layers (intestinal epithelium, epithelium of choroids plexus, Sertoli cells in the testis or lung alveolar epithelial cells) can be utilized to deliver drugs/therapeutics, diagnostics and other cargo substances into the tissue by developing various ligands that cluster the receptors and stimulate their transmigration. These are typically antibodies, but could be peptides, oligosaccharides, etc.

In some instances it may be desirable to select a targeting region having affinity for an epitope capable of causing receptor-mediated endocytosis when bound.

In some instances the cell type of interest will be a cancer cell. In some instances the cell type of interest will not be a cancer cell. In some instances the cell type of interest will be a mammalian cell other than a lung carcinoma cell. In some instances the cell type of interest will be a polarized cell, including without limitation, a brain endothelial cell, a renal endothelial cell, an alveolar lung epithelial cell, and an intestinal epithelial cell. In some instances the cell type of interest will be a non-polarized cell of endodermal, ectodermal, or mesodermal origin.

In some instances the cell type of interest will be a cell capable of undertaking receptor-mediated endocytosis. In some instances the cell type of interest will be a cell capable of undertaking receptor-mediated transcytosis.

In some instances the cell type of interest will be selected to provide a means to transport the cargo substance across a biological barrier, such as the blood-brain barrier or the blood-retinal barrier.

In an embodiment of the invention, subunits capable of forming a pentameric complex (“P5”) comprised of a verotoxin B-subunit mutant scaffold and a variable domain (V_{H}H) of a llama heavy chain antibody (“FC5”) having affinity for a blood brain barrier epitope were produced. This complex pentamerized and showed improved efficiency in binding and crossing the blood brain barrier compared to a monomeric form of the antibody.

In another embodiment of the invention, a pentameric complex (“ES1”) comprised of a verotoxin B-subunit mutant scaffold and a single domain antibody (“sdAb”) having affinity for a carcinoma antigen was produced. This complex pentamerized and was internalized into cells having expressing the antigen (CEACAM6) on their surface.

Similarly, a pentameric complex of single domain antibody AFAI, ES1, against lung cancer antigen showed improved binding and internalized into lung cancer cells in contrast to monomeric form.

The preferred size for the targeting region will generally be determined based on the size of the multimeric complex formed by the multimerization domain. For example, the pentamerized verotoxin B-subunit mutant scaffold described in the examples herein is about 38.5 Kda. This is adequate to permit a targeting region of 14 Kda. For large cargo substances, it may be desired to employ a longer linker sequence to reduce steric hindrance issues. For example, one could readily use a construct such as [(multimerization domain)-(targeting region) - - - X] where - - - is the linker, and X is the cargo molecule of interest and could be up to 100 Kda, 150 Kda or 200 kDa.

In some instances it will be desirable to use a pH sensitive linker or enzymatic cleavable linker capable of releasing the cargo substance after delivery. Other non-limiting examples of linkers that can be used are aldehyde/Schiff base linkage, or suphydryl linkage, or through biotin-avidin technology

The targeting region can be selected based on the cell type of interest and its available antigens. For example, with respect to targeting cargo molecules to cross the blood brain barrier, either or both of FC5 and FC44, described herein, can be used. The detailed method provided in the examples relates to FC5. However it will be understood by those skilled in the art that, where the targeting sequence is an antibody or other polypeptide or protein sequence, one can readily substitute a suitable nucleic acid sequence encoding the targeting region of interest for the nucleic acid sequence of FC5 as described herein, to produce a subunit having the targeting region of interest linked to the multimerization domain.

In some instances it will be desired to use a fragment of an antibody rather than the entire antibody. It will be understood that an antibody fragment need not be actually fabricated from an antibody but may in fact be synthetically manufactured or produced recombinantly to provide the desired sequence. In some instances an antibody fragment may differ significantly from the amino acid sequence of the whole antibody, except with respect to one or more CDR regions which will be preserved to the extent necessary to maintain antigen binding and specificity at tolerable levels.

The exact nature of the targeting region will preferably be selected for optimal suitability for the species of interest. For example, where subunits including an antibody derived targeting region are to be used in delivering cargo substance to human patents, the antibody will preferably be humanized, or a human antibody or fragment thereof will be employed.

In some instances, it will be preferred to select a multimerization domain which is at least 25%, 35%, or 45% as large (by mass) as the targeting domain. In some instances it will be desirable to select a multimerization domain which is no more than 80%, 70%, or 60% as large (by weight) as the targeting domain.

The choice of multimerization domain will depend on the number of subunits desired in the final complex, as well as suitability of the resulting complex for its intended purpose. In some instances the multimerization domain will be a polypeptide sequence derived or derivable from a naturally occurring protein. In some instances the multimerization domain will be a natural or synthetic polypeptide sequence selected to permit enzymatic or non-enzymatic chemical linking of two, three, four or more subunits. In some instances the multimerization domain will be a natural or synthetic polypeptide sequence selected to permit structured aggregation or other non-chemical structured association between two, three, four or more subunits under biologically useful conditions. As used herein, the term “structured association” refers to a relationship between subunits which maintains them in a substantially consistent spatial relationship to one another in a biologically functional form.

By way of example, some multimerization domain which might be of interest for some applications include: verotoxin B-subunit mutant scaffolds, helix-turn-helix peptide oligomerization domains, and the tetramerization domain of p53.

Cargo substances of interest can be linked to subunits by a variety of means, many of which are common in the art. By way of non-limiting example: linking of therapeutics or imaging agents can be performed chemically or using genetic engineering in the form of fusion proteins or linking to nanoparticles that are encapsulated with the drug of interest.

In the example herein, the linkage of P5 to Cy5.5 was obtained using Cy5.5 NHS ester which binds to the lysine amino acid in the P5 in 1:1 Molar ratio. Purification was performed using G-25 columns.

FC5 has also been linked to liposomes encapsulated with doxorubicin. The FC5-liposome complex targets the brain in animals while non-targeted liposomes or free doxorubicin drug don't.

Linkage of the targeting region-multimerization domain complex to agents such as therapeutics can be performed using any number of methods available in the art, based on the functional groups available for attachment in the particular situation. By way of non-limiting example as direct attachment of drug to antibody, or aldehyde/Schiff base linkage, or suphydryl linkage, or acid-labile linkages, or enzymatically degradable linkers or through biotin-avidin technology (Garnett M C. Targeted drug conjugates: principles and progress. Adv Drug Deliv Rev. 2001 Dec. 17; 53(2):171-216.) In addition to this, linking the antibody can be undertaken to functionalized nanoparticles whether they are quantum dots, carbon nanotubes, nanoshells, nanorods, gold nanoparticles, supraparamagnetic nanoparticles etc. Common functionalization procedures include use of hydroxyl groups, carboxyl groups or amine groups.

Nanoparticles as big as 100 nm in size have been successfully delivered using this approach. It is believed that larger cargo materials could also be delivered, such as molecules 150 or 200 nm in size based on the understanding that antibodies capable of causing endocytosis/transcytosis of such large molecules are present on cells.

By way of non-limiting example, cargo molecules of potential interest include one or more of: neuropeptide Y, superoxide dismutase, parathyroid hormone, adrenocorticotropic hormone, adenosine deaminase, ribonuclease, alkaline phosphatase, angiotensin, antibodies, arginase, arginine deaminease, asparaginase, tissue plasminogen activator, calcitonin, chemotrypsin, cholecystokinin, clotting factors, dynorphins, endorphins, enkephalins, erythropoietin, gastrin-releasing peptide, glucagon, hypothalmic releasing factors, interferon, non-naturally occurring opioids, oxytosin, papain, prolactin, soluble CD-4, somatostatin, somatotropin, thyroid stimulating hormone, vasopressin, and analogues of such peptides, as well as other suitable enzymes, hormones, proteins, polypeptides, enzyme-protein conjugates, siRNA etc.

In addition, for brain-related therapies there will be instances where it is desired to have a cargo molecule comprising one or more potent neuropharmaceuticals that don't cross the BBB. By way of non-limiting example, classes of major interest are oncology in brain cancers (involving cargo molecules such as anti-EGFR antibodies), neurodegenerative diseases such as Alzheimer's disease (cargo molecules including: Abeta amyloid antibodies or peptides), stroke (cargo molecules including: multiple neurotrophins), brain injury (cargo molecules including: BDNF, FGF-2), Parkinson's disease (cargo molecules including: GDNF, glutathione), Amyotrophic lateral sclerosis (cargo molecules including: BDNF, CNTF), lysosomal storage disorders of the brain (cargo molecules including lysosomal enzymes), schizophrenia (cargo molecules including Neuregulin-1), and depression (cargo molecules including BDNF).

In addition, in some instances, cargo molecules of interest will include small molecules. These can be either directly attached or encapsulated in functionalized nanoparticles which then can be linked to the subunit. Doxorubicin and paclitaxel are examples of potent chemotherapeutic agents of potential to treat brain cancer which can be delivered in this manner.

In an embodiment of the invention the targeting region comprises a polypeptide sequence comprising at least 90 amino acids including at least one or two of the following three contiguous amino acid sequences: KNLMG, TISGSGGTNYASSVEG, and AFAI.

With reference to the specific example of FC5 as a targeting agent, the sequence employed is shown below. One or more of the underlined bold amino acid sequences in FC5 comprising the CDR regions of the antibody are believed to play a role targeting in FC5 targeting the blood brain barrier. In some instances, it will be desired to include 2 or three of these sequences in the targeting agent. It will be understood that considerable sequence variation outside the CDR regions can be tolerated, while less variation can be tolerated within the CDR regions. In some instances, a sequence at least 85%, 90%, 95%, 98%, 99%, or 100% identical to the CDR regions of SEQ. ID. NO. 1 and at least 50%, 60%, 70%, 80%, 85%, 90%, 95%, 97%, 98%, or 99% identical to the non-CDR regions of SEQ. ID. NO. 1 will be preferred.

SEQ. ID. NO. 1

EVQLQASGGG LVQAGGSLRL SCAASGFKIT HYTMGWFRQA
PGKEREFVSR ITWGGDNTFYSNSVKGRFTI SRDNAKNTVY
LQMNSLKPED TADYYCAA GSTSTATPLRVD Y WGKGTQVTVSS

### The P5 Amino Acid Sequence:

SEQ. ID. NO. 2

stpdcvtgkveytkyndedtftvkvgdkelftnranlqslllsaqitgmt
vtiktnachngggfsevifrGGGGSGLAGSEVQLQASGGGLVQAGGSLRL
SCAASGFKITHYTMGWFRQAPGKEREFVSRITWGGDNTFYSNSVKGRFTI
SRDNAKNTVYLQMNSLKPEDTADYYCAAGSTSTATPLRVDYWGKGTQVTV
SS EQKLISEEDLNHHHHH

As written above, the italic lower-case is the verotoxin sequence; the upper case is the linker; the underlined is the FC5 sequence; and, the italic upper case is the Myc-His tag.

With further reference to the example employing FC5 as the targeting agent, the full sequence of the P5 construct employed is:

SEQ. ID. NO. 3

GTTNCGANTGNNTNGAGGGTAGAATTCATGAAAAAAANCGCNATCGCGAT
CNNGTTGCCTTGNCTGGTTTCGCTNCCGNTGCGCAGNCCGNCTTCGTACN
ATCCGGGCCCGGCAGGCGGCATCCGGTGGCGGCGGTTCCACGCNTGATTG
TGTAACTGGTAAGGTGGNGTATACAAAATATAATGATGAAGATACCTTTA
CAGTTAAAGTGGNNGATAAAGAATTATTTACCAACAGAGCGAATCTTCAN
NCTCTTCTTCTCAGTGCGCNAATTACGGNGATGACTGTAACCATTAAAAC
TAATGCCTGNCATAATGNAGGGGGATTCAGCGAANTTATTTTTCGTGGCT
GGAGGTAGGTTCCNGAGATGTGCAGCTGCAGGCGTCTGGAGGAGGATTGG
TGCAGGCTGGGGGCTCTCTGAGACTCTCCTGTGCAGCCTCTGGATTCAAA
ATCACTCACTATACCATGGGCTGGTTCCGCCAGGCTCNAGGGAAGGAGCG
TGAATTTGTATCACGTATTACTTGGGGCGGTGATAACACCTTCTATTCAA
ACTCCGTGAAGGGCCCATTCACCATTTCCAGAGACAACGCCCAGAACACT
TTNTATCTNCAAATGAACANCCTGNACCTNAGGACACGGCCGATTNTTAC
TGTGCANCACGNTCGACGTNCACTGCGACNCCNCTTAGGGTGGACTACTG
GGGCAAAGGACCCAGGTCACCGTCTCCTCAGGANCCAACAAANCTGATCC
GCGANGAANATCTGACTNTCNCCATCACNNTTAGTGAANCTNGNACTGGC
CGCGTTTACAACGTCNNGCTGGAAACCCTNCG

(The underlined sequence represent the FC5 incorporated within the pentamer sequence.)

### Sequence of FC5

SEQ. ID. NO. 4

GAGGTCCAGCTGCAGGCGTCTGGAGGAGGATTGGTGCAGGCTGGGGGCTC
TCTGAGACTCTCCTGTGCAGCCTCTGGATTCAAAATCACTCACTATACCA
TGGGCTGGTTCCGCCAGGCTCCAGGGAAGGAGCGTGAATTTGTATCACGT
ATTACTTGGGGTGGTGATAACACCTTCTATTCAAACTCCGTGAAGGGCCG
ATTCACCATTTCCAGAGACAACGCCAAGAACACGGTCTATCTGCAAATGA
ACAGCCTGAAACCTGAGGACACGGCCGATTATTACTGTGCAGCAGGTTCG
ACGTCGACTGCGACGCCACTTAGGGTGGACTACTGGGGCAAAGGGACCCA
GGTCACCGTCTCCTCA

By way of non-limiting example, therapeutic targets having known antigens which bind to them, thereby enabling use of the targeted multimer approach taught herein, include: Alzheimer's (amyloid), cancer (EGFR, platelet-derived growth factor receptor, P53, VEGFR) cardiovascular diseases (nitric oxide, endothelin), Parkinson's disease (GDNF, dopamine, dopamine receptors), inflammation such as asthma (integrins, cytokines, TNF alpha, chemokines).

In some instances it will be desirable to combine the methods and compounds disclosed herein with other therapeutic approaches. For example, to enhance small molecule drug delivery to the brain lipidation is commonly used. Such an approach will not always be preferred however, as this can result in adverse effects of increased drug concentration in other organs as well. Invasive methods for drug delivery are also well documented such as local drug administration using intracerebral or intracerebroventricular injection or osmotic blood-brain barrier opening in brain tumor patients. However, this approach will not be suitable for chronic brain diseases such as Alzheimer's disease.

The subunits can be provided in pharmaceutically acceptable formulations using formulation methods known to those of ordinary skill in the art. Where the subunits further include a cargo molecule and are ready for administration to a patient, they will typically be formulated to permit easy delivery with limited preparation. For example, these formulations can be administered by standard routes. In general, the combinations may be administered by the topical, transdermal, intraperitoneal, oral, rectal or parenteral (e.g., intravenous, intraspinal, subcutaneous or intramuscular) route.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

In certain embodiments, subunits further including cargo substance may be orally administered, for example, with an inert diluent or an edible carrier. The subunits with bound cargo substance (and other ingredients, if desired) may also be enclosed in a hard or soft shell gelatin capsule, or compressed into tablets. For oral therapeutic administration, subunits with bound cargo substance may be incorporated with excipients and used in the form of ingestible tablets, buccal tablets, capsules, elixirs, suspensions, syrups, and the like. To administer a compound of the invention by other than parenteral administration, it may be necessary to coat the compound with, or co-administer the compound with, a material to prevent its inactivation. Osmotic minipumps may also be used to provide controlled delivery of high concentrations of subunits with cargo substance through cannulae.

In certain embodiments, subunit-cargo molecule complexes may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

In some instances, it will be desirable to provide subunits without attached drug but in a condition that permits ready linking of the drug to the subunit for administration. In such cases, it will sometimes be desirable to provide a kit comprising, a multiplicity of subunits having a multimerization domain and a targeting region of interest, together with instructions for linking the subunits to a cargo material of interest. In some instances a purification means such as an affinity column will also be provided. In some instances the subunits may be in freeze dried form and instructions for reconstitution into a suitable aqueous form will be provided.

In some instances, formulations will be selected for suitability in use of detecting antigens of interest in tissue sections or biopsies of diseased organs for the purpose of diagnosing the disease, determining the modality of disease (e.g., cancer expressing specific antigens; brain tumor or epileptic brain tissue expressing drug resistance transporters, etc) or determining the therapeutic approach suitable for the modality of disease (e.g., drugs that evade specific multidrug transporters; chemosensitivity, etc.).

In an embodiment of the invention there is provided a method of obtaining information useful in the diagnosis or treatment of a brain disease in a human or other mammalian patient. The method comprises administering to the patient a plurality of subunits comprising a targeting region having affinity for a blood-brain barrier antigen, a multimerization domain, and a cargo substance including a diagnostic agent. In some instances the diagnostic agent may be a labeled or unlabeled peptide, polypeptide, small molecule, and/or oligosaccharide having affinity for or being a substrate of a cell component implicated to be involved in or indicative of a disease state. In some instances the diagnostic agent may be a molecule which would be expected to be the subject of unusual diffusion, segregation and/or transport in a disease state.

In an embodiment of the invention there is provided the use of a targeting region joined to a multimerization domain in the manufacture of a medicament. In an embodiment of the invention there is provided the use of a subunit comprising a targeting region, a multimerization region and a cargo molecule in the manufacture of a medicament, for example for treatment of a brain, renal, ocular, or intestinal disorder.

In an embodiment of the invention there is provided the use of a multimerization domain to enhance uptake of a cargo substance into a cell.

In an embodiment of the invention there is provided the use of a multimerization domain to enhance receptor-mediated transcytosis of a cargo substance.

### Multimeric Display of FC5 Increases Binding to Brain Vessels

FC5 is a llama single-domain antibody selected from a naïve phage display library of llama single-domain antibodies. FC5 was selected by differential panning between lung and brain human microvascular endothelial cells as a selective ‘binder’ to brain microvascular endothelial cells. FC5 has been shown to internalize into human brain endothelial cells by a process of receptor-mediated endocytosis. FC5 can also transmigrate human brain endothelial cell monolayer (i.e., blood-brain barrier) by a process of transcytosis.

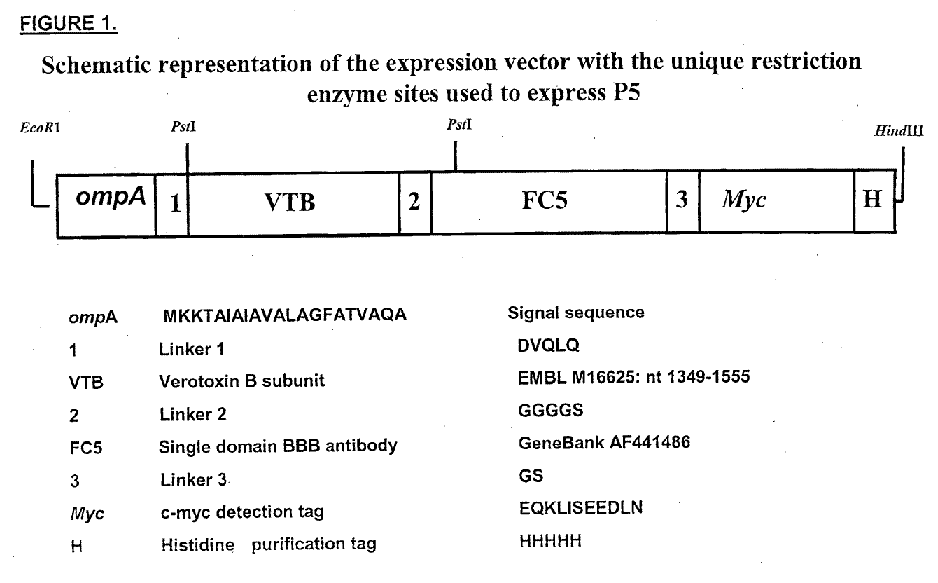

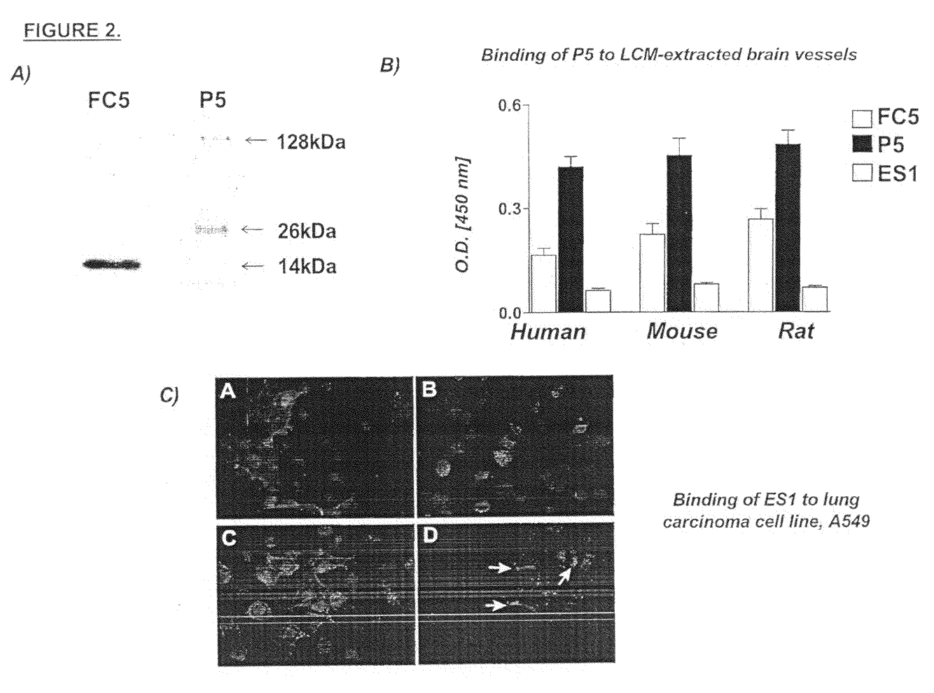

Pentamerization of FC5 on VT1B was performed to introduce avidity in FC5. The construct design is shown in FIG. 1. The molecular weight (128 kDa) of the purified pentameric construct P5 was confirmed on a Western blot (FIG. 2A). 26 kDa band (FIG. 2A) corresponds to a single FC5 fused with VT1B.

The binding of P5 to LCM-extracted brain vessels from human, mouse and rat was compared to that of either monomeric FC5 or unrelated pentameric sdAb, ES1. P5 sdAb showed higher binding to brain microvessels than monomeric FC5 (FIG. 2B). Unrelated pentameric antibody ES1 (constructed by pentamerizing sdAb against lung carcinoma antigen, CEACAM6) showed only negligible binding to immobilized brain vessels (FIG. 2B). Although FC5 was selected by panning against human CEC, it also recognized BBB-selective antigens in brain microvessels of other species, including rat and mouse (FIG. 2B). ES1 pentameric antibody against CEACAM6, showed strong and selective binding and internalization into the lung carcinoma cell line, A549, whereas monomeric form bound to cells weakly and was not able to internalize (FIG. 2C). AFAI and its pentameric antibody form were characterized in terms of their functional affinity to A549 cells, internalization and translocation after cell binding. These data revealed the importance of internalization as a functional parameter of the activity of pentameric antibodies.

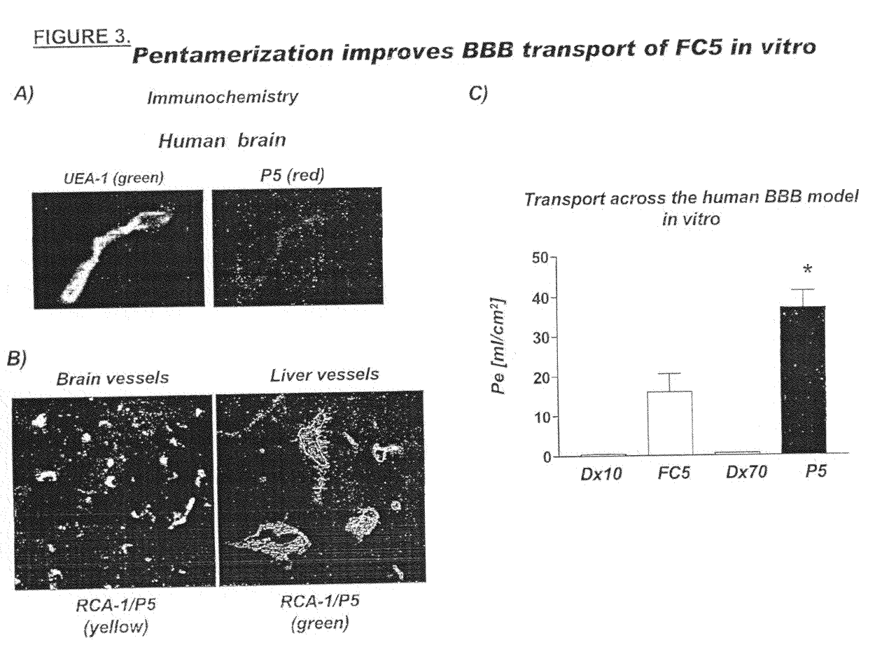

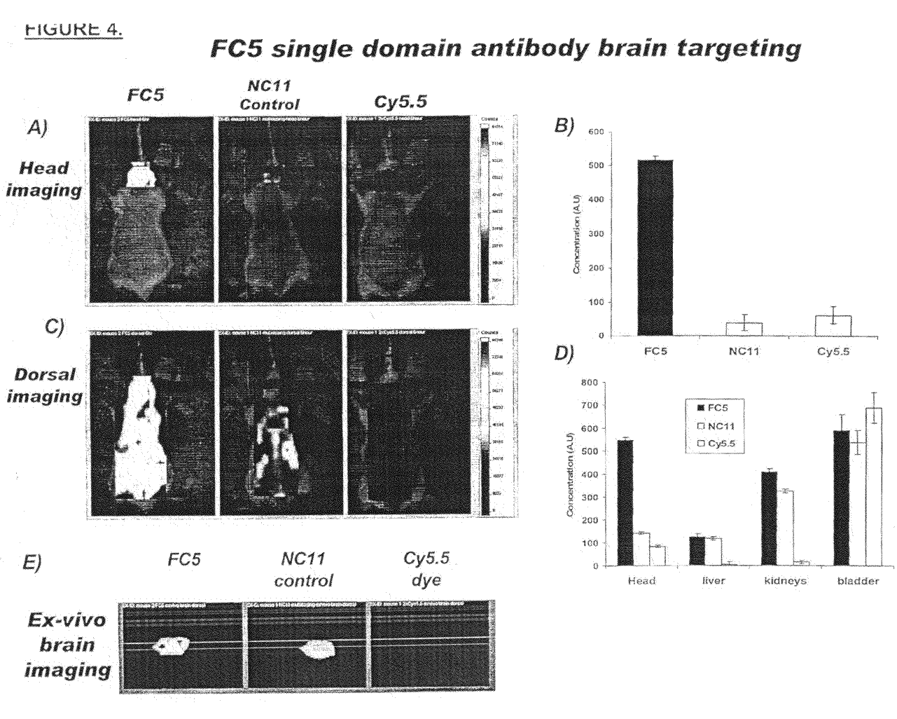

Immunohistochemistry also demonstrated strong vascular staining by P5 in brain sections —P5 immunoreactivity co-localized with vascular structures stained with lectins UEA 1 and RCA 1 in human (FIG. 3A) and rat brain (FIG. 3B). In contrast, P5 failed to stain vessels in rat liver sections (FIG. 3B), indicating that antibody recognizes antigens selectively expressed in brain vessels.

### Multimeric Display of FC5 Increases Transport Across In Vitro BBB

The P5 underwent a rapid internalization into cultured human CEC within first 15 min of addition. P5 also exhibited higher degree of transendothelial migration [i.e., significantly higher permeability coefficient Pₑ)] when compared to monomeric FC5 across human brain endothelial cells (FIG. 3C). The transendothelial migration of P5 was also faster (i.e., seen as early as 5 min after the addition to the luminal chamber of the BBB model) compared to that of FC5 (seen only after 15 min). Unrelated pentamerized sdAb ES1 did not show measurable migration across CEC monolayer. Neither 10 kDa nor 70 kDa dextran migrated across the same membranes.

In summary, P5 binding to LCM-captured brain vessels was 2-fold higher than that of monomeric FC5. Similarly, P5 uptake into HCEC and its transport across in vitro BBB models were faster when compared to monomeric FC5, indicating that antigen cross-linking is important in initiating endocytosis/transcytosis in CEC, similar to prior observations in other cells. To exclude the involvement of VT1B subunit in binding and transmigration of P5, a mutant VT1B subunit that lacks the capability to bind Gb3 receptor was used. A non-related pentameric antibody (ES1) failed to either bind to brain vessels or transmigrate across human CEC.

The following results show that multimerization of single-domain antibodies achieves superior targeting in in vivo (animal model) systems.

### P5 is More Efficient in Targeting the Brain In Vivo Compared to FC5

Optical Imaging Studies

Since sdAbs have no available —SH groups for conjugation with therapeutic moieties, FC5 was engineered to express an additional free cysteine.

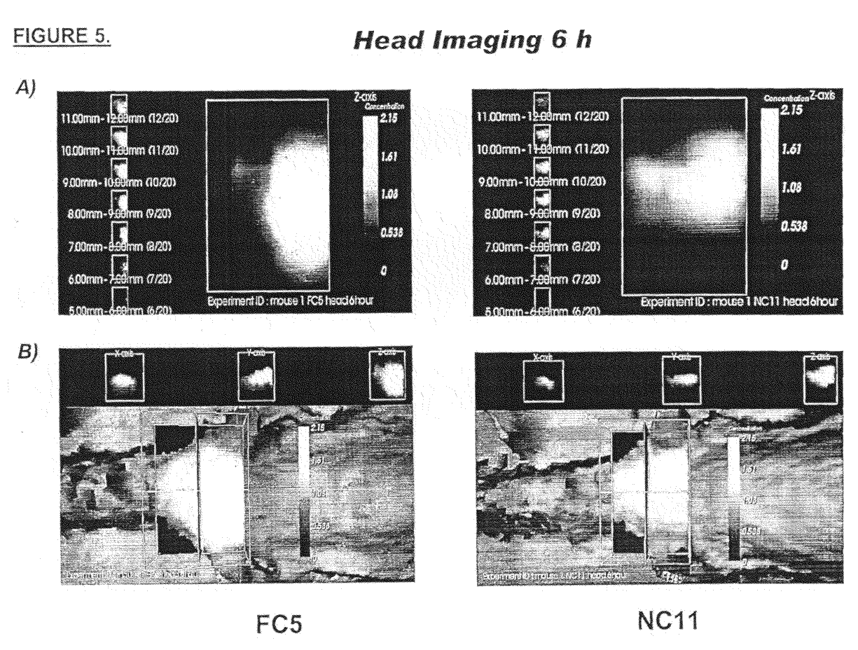

FC5 was conjugated with the near-infrared probe, Cy5.5, through NHS ester linkage and injected in mice intravenously via the tail vein. Optical imaging using eXplore Optix small animal imager (670 nm excitation laser) 6 hour after injection showed higher accumulation of the FC5 in the head region compared to the negative control single-domain antibody, NC11, isolated from the same library against different target (FIG. 4). Quantification of the fluorescence concentration using OptiView software in various regions, including head (FIG. 4, B&D) showed a selective accumulation of FC5 in the head. Ex-vivo imaging of brains removed from animals after kill perfusion (FIG. 4E) demonstrate higher fluorescence accumulation in the brain of FC5-injected animals compared to those injected with NC11. Optiview software analysis allows for coupling of depth and concentration of the fluorescence marker. The depth/concentration analysis (FIG. 5A) shows that FC5 fluorescent signal originates from the depth of 6 mm to 10 mm brain slices, whereas NC11 fluorescent signal is weak and similar in all slices. Topographic representation and 3D reconstruction of the animals confirmed the FC5 fluorescence preferentially in the deep head volume planes while faint NC11 fluorescence was similar in all volume planes (FIG. 5B).

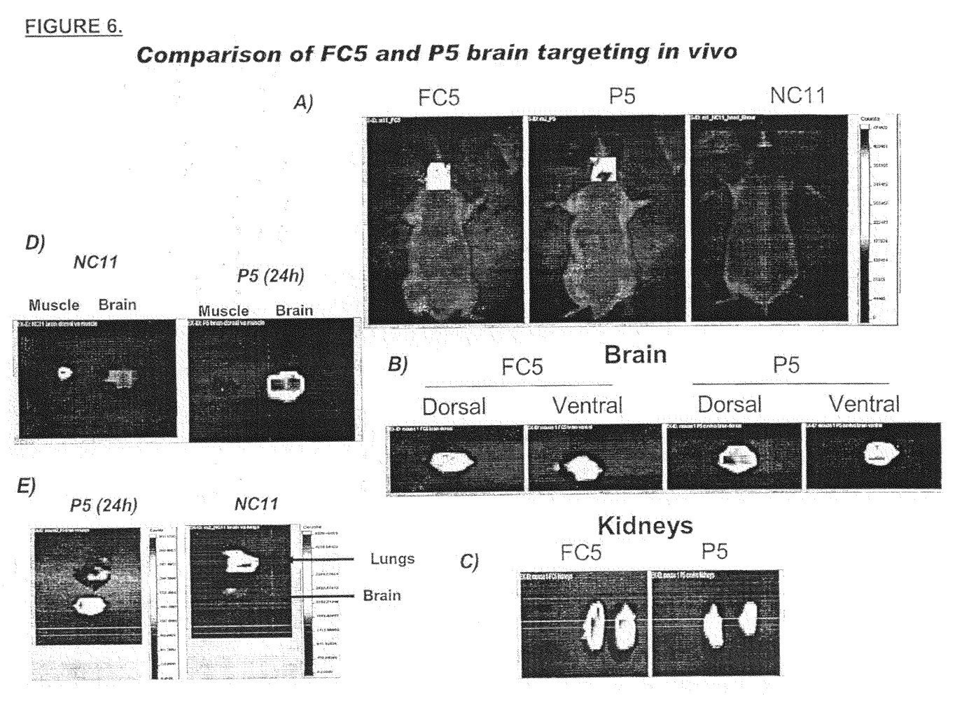

FIG. 6A shows animals injected with equimolar amounts (50 μg of FC5 or 250 μg of P5) of either Cy5.5-labeled P5, FC5 or NC11 and imaged after 6 hours. P5 shows higher fluorescence intensity signal in the head compared to the FC5 (FIG. 6A). Similarly, ex-vivo brain imaging shows higher brain fluorescence in P5-injected animals (FIG. 6B). Kidney levels for the monomeric FC5 (13 Kda) were higher than the pentameric form P5 (126 kDa) (FIG. 6C) likely due to higher kidney clearance of small molecular weight FC5. To demonstrate the specificity of the P5, ex-vivo imaging was performed on lungs, muscle and brain of animals injected with P5; in all cases fluorescence signal of P5 was strongest in the brain (FIGS. 6D&E).

Confocal Laser Microscopy Studies

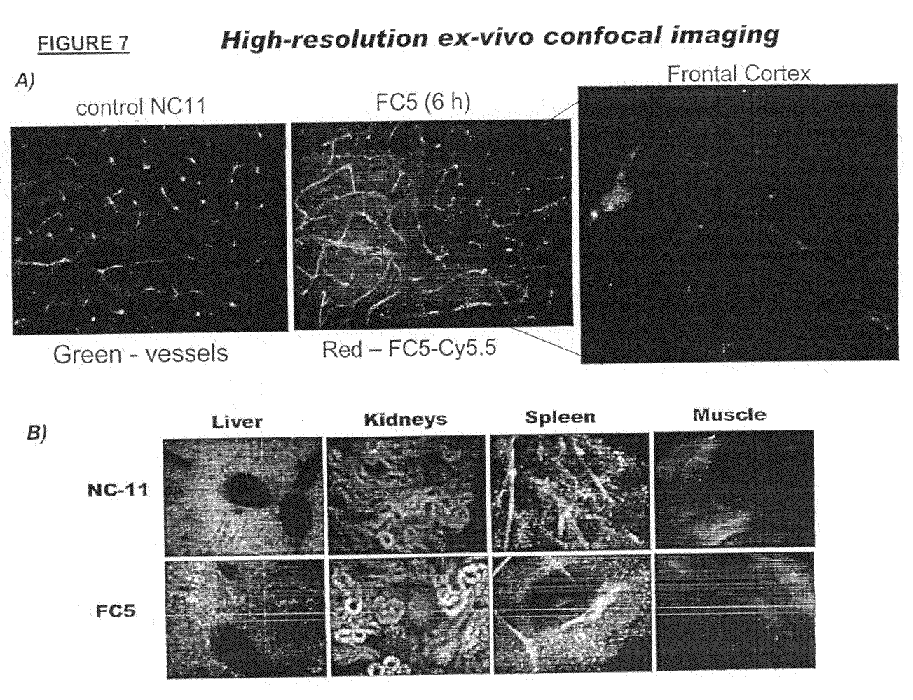

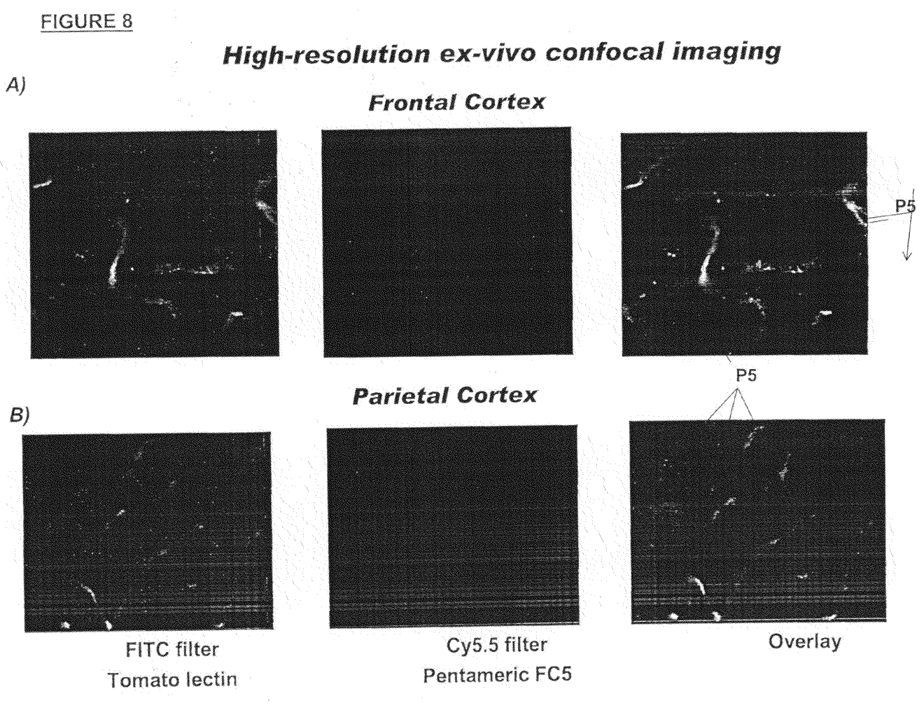

After being subjected to in vivo optical imaging, animals were injected with Tomato lectin-FITC to stain brain vessels, perfused, and their brains were sectioned. The brain sections from animals injected with Cy5.5-labeled FC5, P5 or NC 11 were observed under the microscope using near-infrared filters. FIG. 6A shows that FC5, but not NC11 is detected by fluorescence microscopy in brain vessels and in brain parenchyma (arrows) in the frontal cortex. Pattern of FC5 staining shows vesicular staining throughout the depth of the brain vessel, resembling endocytotic vesicles. FIG. 7B shows that NC11 and FC5, in contrast to the brain, are localized similarly in other organs. Similarly, high resolution confocal microscopy of brain sections of animals injected with P5 shows the presence of P5 in both brain vessels and the brain parenchyma in the frontal (FIG. 8A) and the parietal (FIG. 8B) cortex.

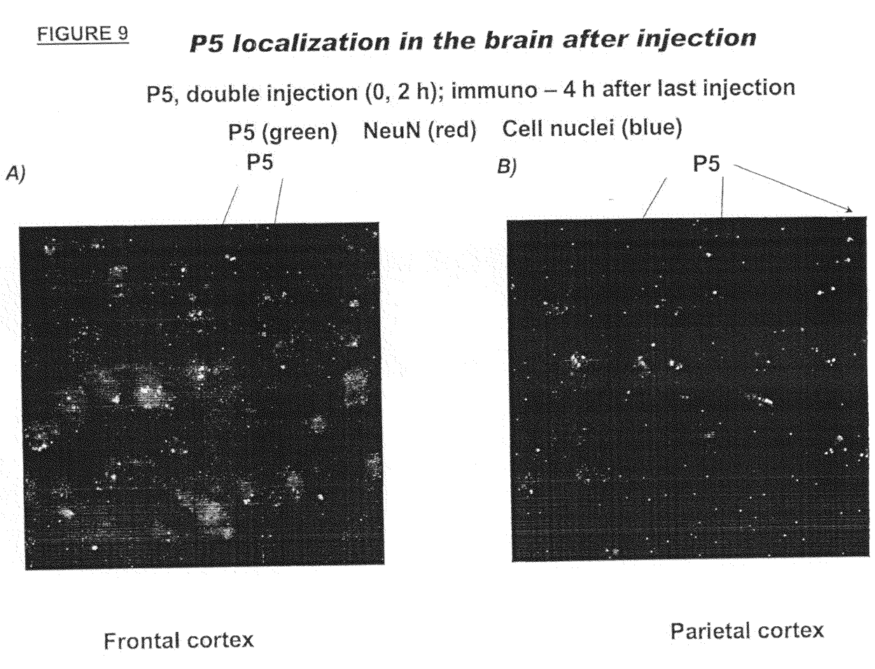

To investigate where P5 localizes after crossing the blood-brain barrier into the brain parenchyma, brain sections of P5-injected animals were immunostained for neuronal-specific nuclear marker, NeuN. FIG. 9 shows co-localization of P5 with NeuN and brain vessels in sections of the fronat and parietal cortex, suggesting that P5 is preferentially taken up by neurons after crossing the blood-brain barrier in vivo.

Proteomics Studies

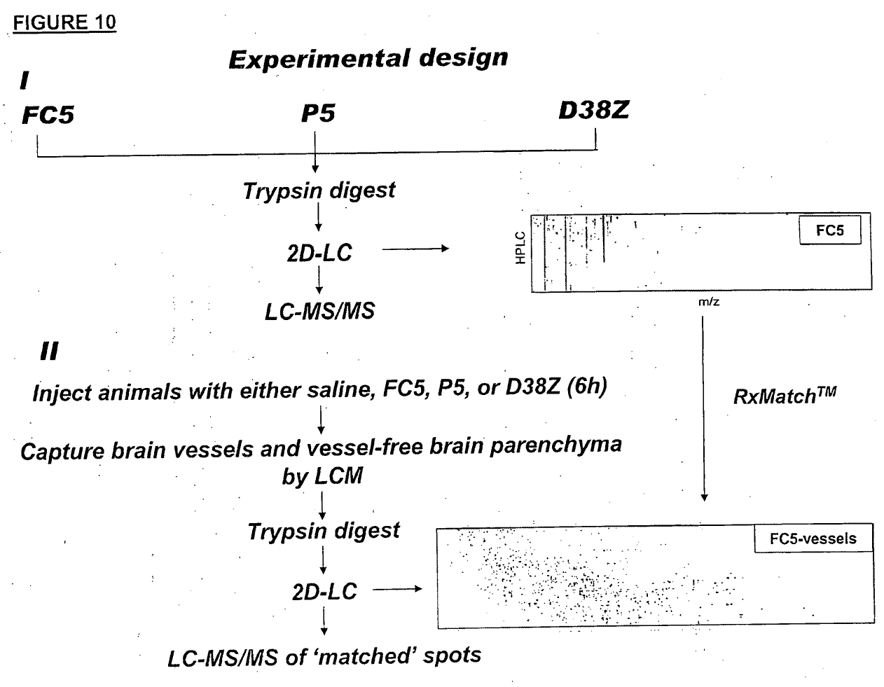

Since animal imaging and confocal microscopy studies relied exclusively on the detection of Cy5.5 probe attached to the antibodies, to unambiguously confirm the presence of antibodies themselves in the brain tissue, detection and sequencing of antibodies were done by proteomics. The experimental design of these studies is shown in FIG. 10. FC5, P5 and control single domain antibody, D38Z were trypsin-digested and their respective 2D maps of all ionizable peptides were generated by 2D-LC, and subsequently sequenced by LC-MS/MS. Animals were then injected with either saline, or equimolar doses of FC5, P5 or D38Z for 6 h; animals were then perfused, their brains removed and sectioned. Brain vessels and vessel-free brain parenchyma were captured using laser-capture microdissection (LCM) microscopy as described in Methods. Each LCM-captured sample was trypsin-digested and 2D maps of all ionizable peptides in these samples were generated by 2D-LC. A custom developed software, RxMatch™ was used to find (‘match’) signatures (specific peptides) of injected single domain antibodies on the 2D maps of all peptides in LCM samples. Since injected antibodies originated from llama, their peptide signatures are not present in mice. ‘Matched’ peptide spots were then sequenced to confirm the identity of peptides as those belonging to respective single domain antibodies.

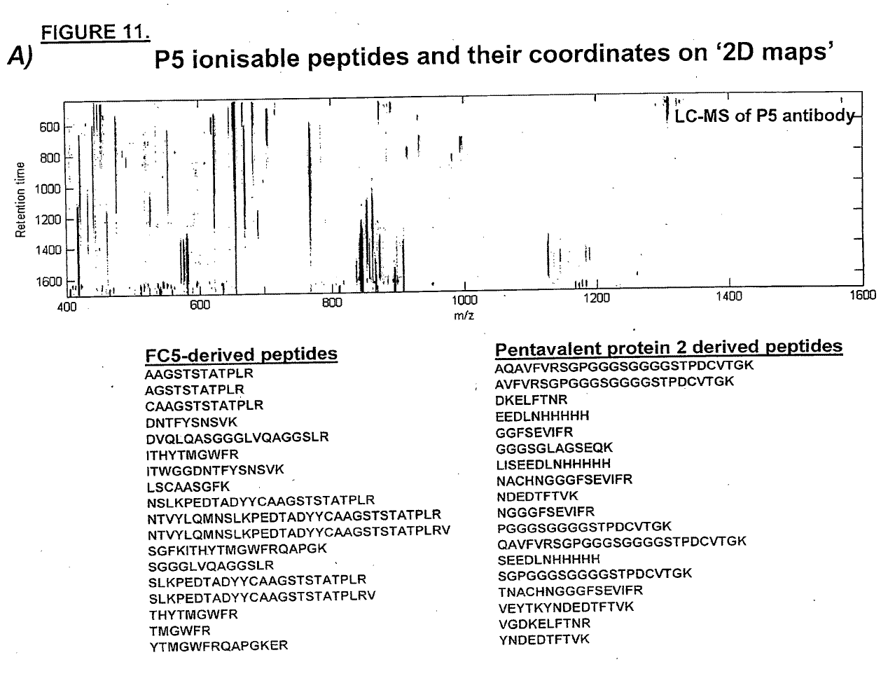

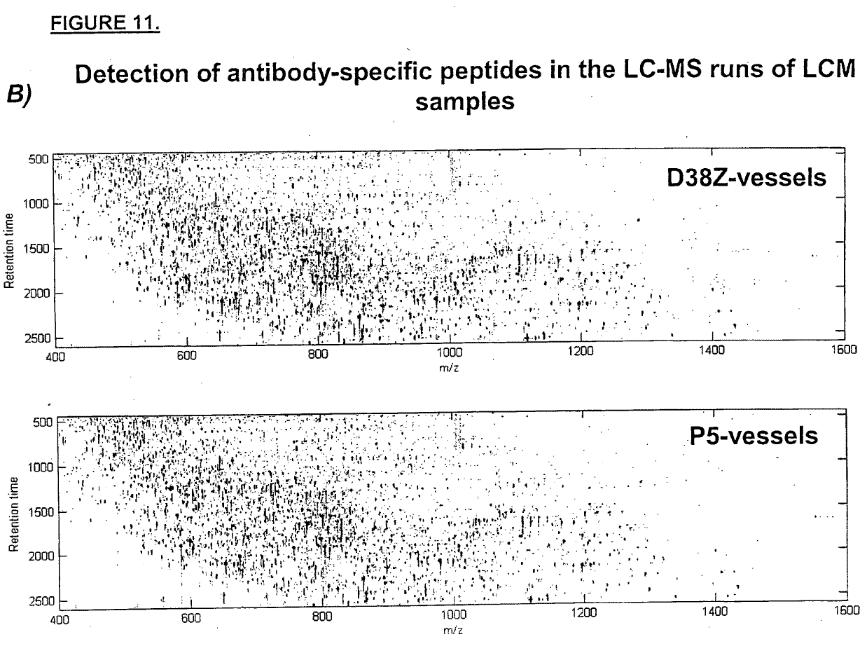

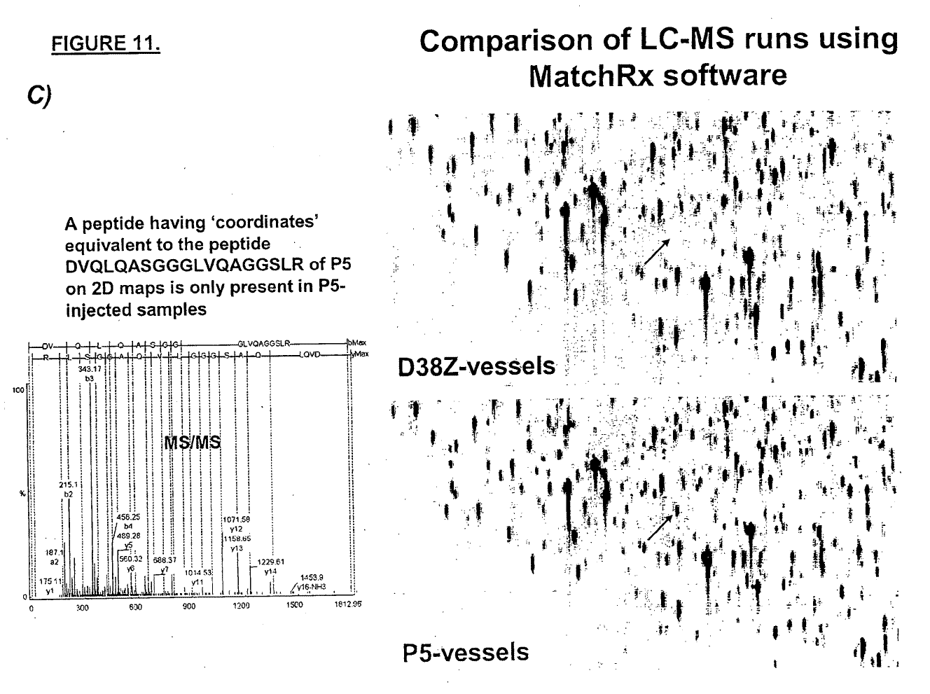

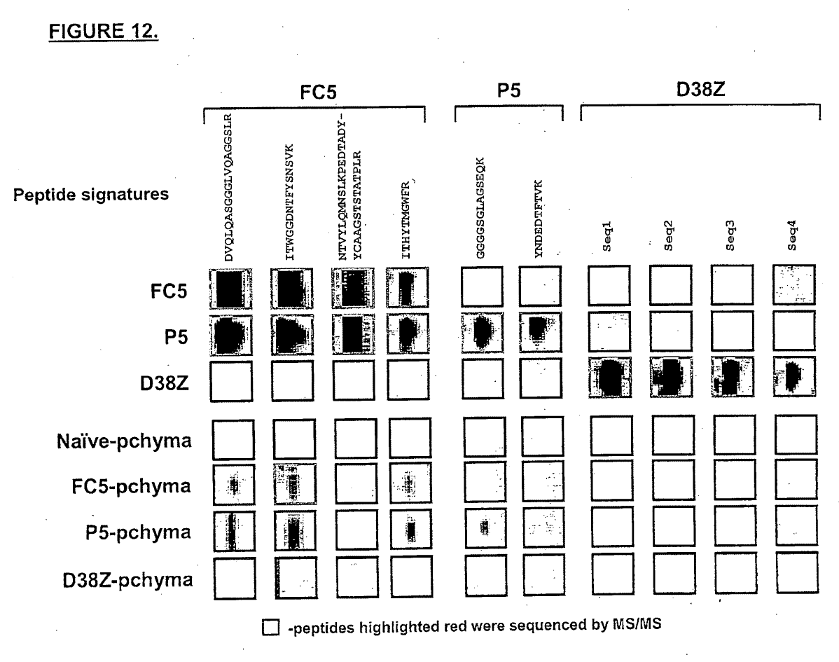

FIG. 11A shows an example of ionizable peptides of FC5 and P5 and the coordinate map of P5. FIG. 11B shows 2D maps of all ionizable in the LCM-extracted vessels of animals injected intravenously with either D38Z control sdAb or with P5. FIG. 12C shows identification of P5-specific peptide in brain vessels of P5-injected animal but not in brain vessels of D38Z-injected animal and confirmation of its identity by sequencing.

FIG. 12 summarizes the results of animal studies where mice were injected with either FC5, P5 or D38Z. No llama antibody-specific peptides were present in either naïve mouse brain (saline-injected animals) or in brain parenchyma of D387-injected animals. However, FC5- and P5-specific peptides were identified (and confirmed by sequencing) in the brain parenchyma of FC5, and P5-injected animals (FIG. 12). Signals of P5 peptides were stronger than those of FC5 peptides. These data confirm that the antibodies FC5 and P5 cross the BBB and their sequences are present in the vessel-free brain parenchyma. Quantitation of the amounts of FC5 and P5 in LCM samples by LC-MS/MS (Table 1) shows that more P5 than FC5 is delivered into the brain after systemic injection.

Pharmacokinetics Studies

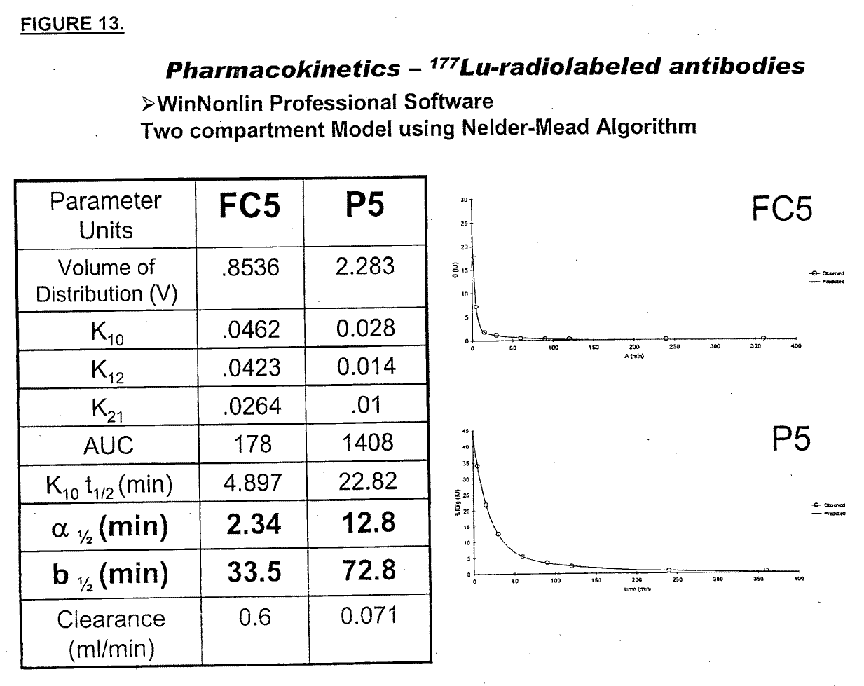

P5 and FC5 were radiolabeled with Lutetium (Lu177) and injected in mice intravenously via tail vein. Blood samples at different time points and the radioactivity in the blood drawn at various time points was determined in gamma counter. Pharmacokinetics analysis was performed using two-compartment model (Nelder-Mead algorithm) (winNonlin Professional software). FIG. 13 shows various pharmacokinetic parameters for FC5 and P5′. Results indicate slower renal clearance and longer plasma half life of P5 compared to the monomeric FC5.

### Multimerization by PEGylation

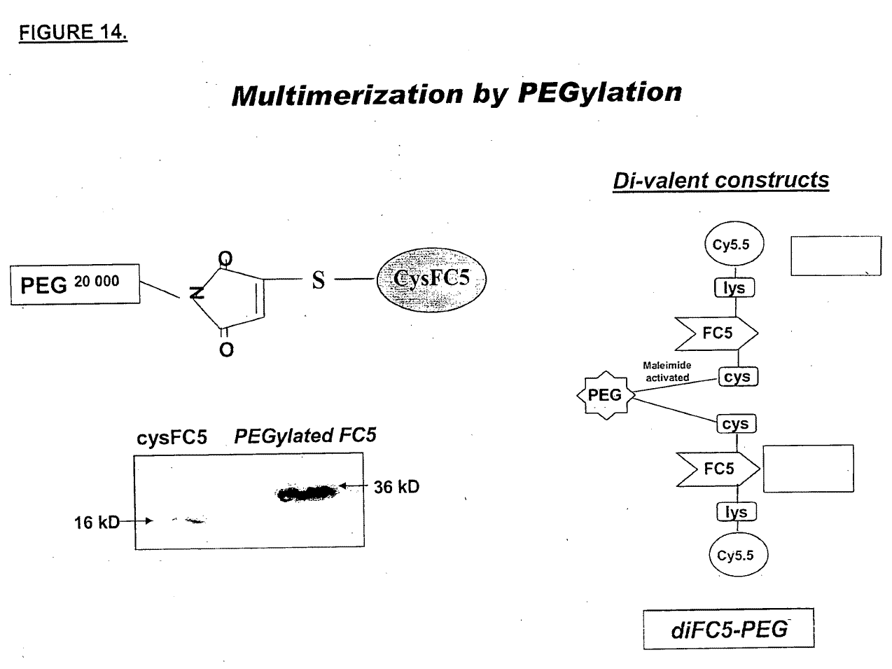

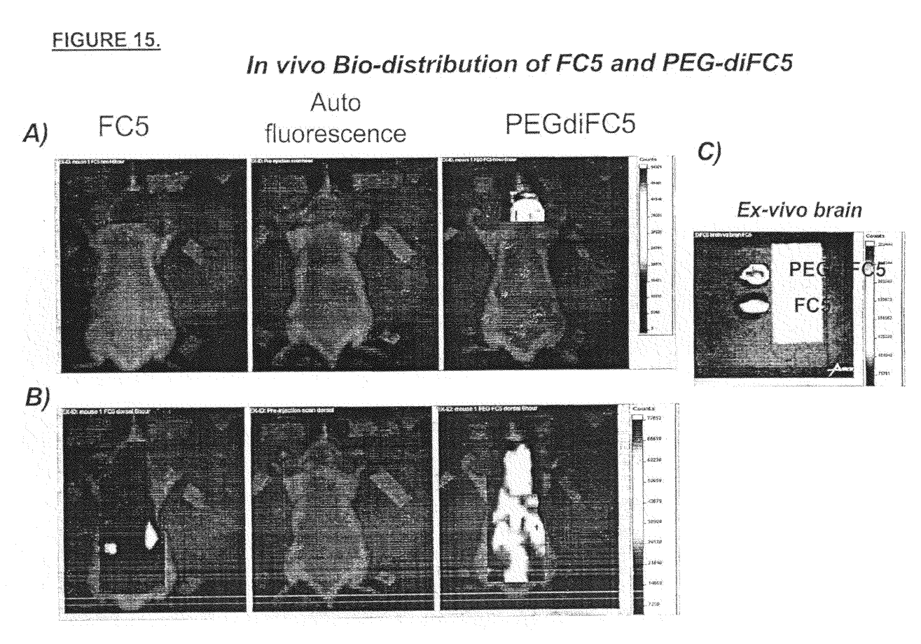

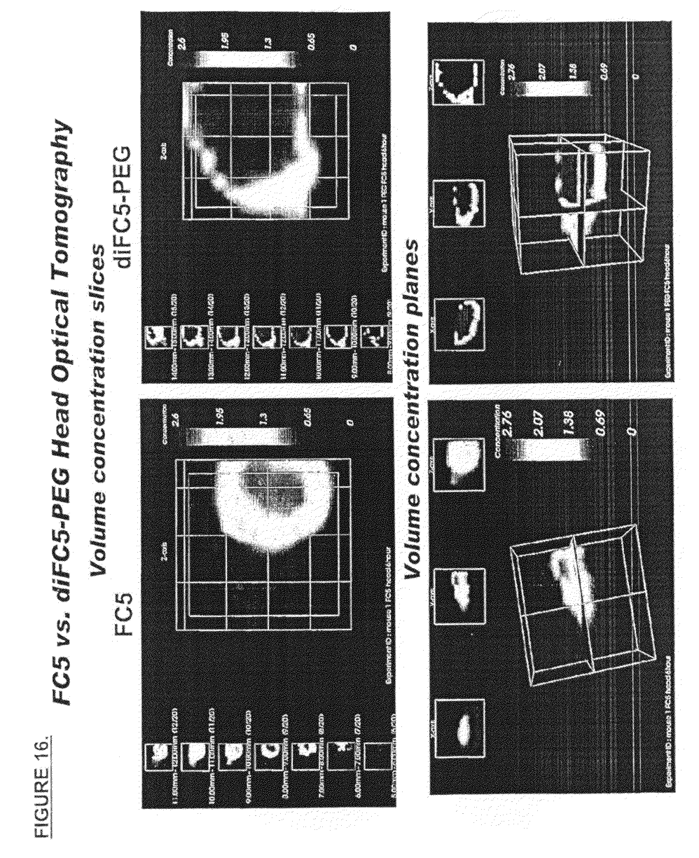

To further explore the efficacy of multimerization in increasing targeted brain delivery of FC5, we used an alternative technique. Polyethylene glycol (20 kD) was linked to cysFC5 via a site-specific linkage to free cysteine residue (FIG. 14). Successful PEGylation of cysFC5 was shown on a Western blot, where PEGylated product shows MW of 36 kD. Subsequently, a di-valent construct of FC5 was generated using bis-functional PEG (diFC5-PEG), whereby two Cy-5.5-labeled cysFC5 were linked to bis-functional PEG (FIG. 14). Brain delivery of equimolar concentration of Cy5.5-FC5 (50 μg) and diFC5-PEG (176 μg) injected i.v. into mice was then compared by optical imaging. Comparative optical imaging of FC5 and diFC5-PEG (FIG. 15 A&B)) shows superior brain delivery of diFC5-PEG. Ex-vivo brain imaging after perfusion also confirmed the higher accumulation of diFC5-PEG in the brain (FIG. 15C). Topographic representation and depth/concentration analyses (FIG. 16) show higher concentration of diFC5-PEG compared to FC5 in the brain at the same depth.

### Multimerization in the Context of Liposomes

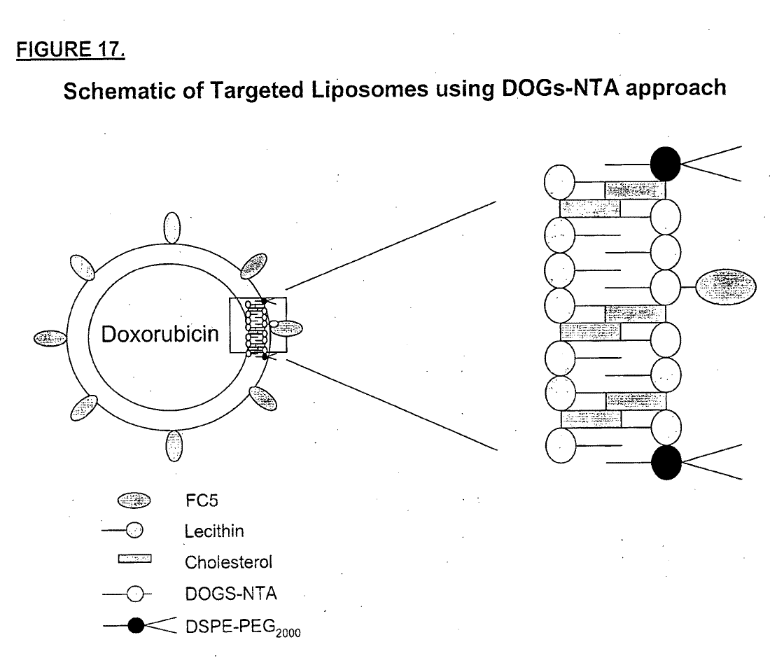

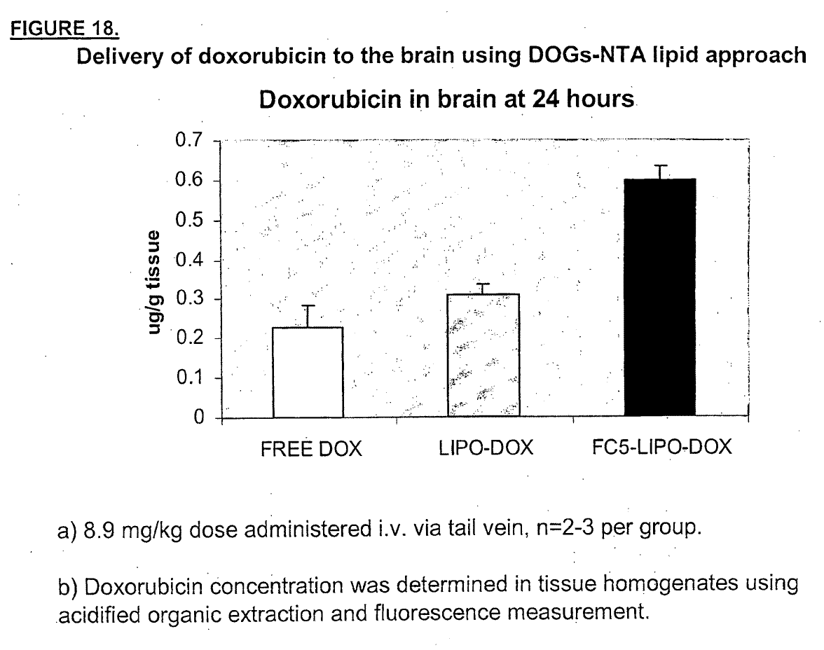

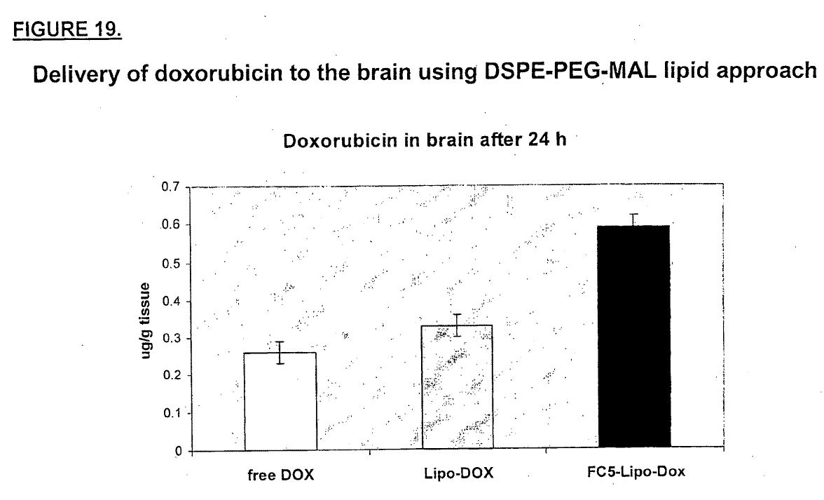

Targeting antibodies can be used to functionalize liposomes or other nanoparticles to deliver therapeutic or imaging payloads. Functionalization of liposomes or nanoparticles with single domain antibodies, such as FC5, could also increase their valency, as multiple number of antibodies can be linked to these nanocarriers. To demonstrate efficacy of FC5 targeted liposomes in delivering drug-payloads, FC5 targeted pegylated liposomal doxorubicin were constructed (as described in Methods) using DOGS-NTA approach where the antibody with the histidine tag spontaneously binds to the NTA in the lipid as shown schematically in FIG. 17. The efficacy of this formulation to deliver doxorubicin to the brain after i.v. injection was compared to that of non-targeted pegylated liposomal doxorubicin or free doxorubicin. Brain levels of FC5-targeted liposomal doxorubicin showed 100% increase compared to the non-targeted liposomal doxorubicin and 200% increase compared to the free drug (FIG. 18). Doxorubicin delivery to the brain was also performed using DSPE-PEG-mal lipid approach where the Cystine FC5 antibody was reduced and then linked to the activated PEG in the liposomal formulation. FIG. 19 shows that Brain levels of FC5-targeted liposomal doxorubicin also showed 100% increase compared to the non-targeted liposomal doxorubicin and 200% increase, compared to the free drug.

The pentameric form of FC5 (P5) displays better binding to brain endothelial cells and vessels and more efficient endocytosis/transcytosis across brain endothelial cells than the monomeric form (FC5).

The pentameric form of FC5 (P5) and di-meric form of FC5 (diFC5-PEG) cross the blood brain barrier in vivo more efficiently than the monomeric FC5 and is co-localized with neurons in the brain tissue.

The multimeric formats of antibodies targeting the brain are more efficient than monomeric formats in carrying therapeutic or imaging payloads across the blood brain barrier.

### Methods

The sdAb AFAI was isolated from a phage display panning of naïve single domain library on non-small cell lung carcinoma cells (Zhang, Li et al. 2004). It was pentamerized with a penabody technology (Zhang, Tanha et al. 2004), generating a penameric single domain antibody ES1. (Zhang, J., Q. Li, et al. (2004). “A pentavalent single-domain antibody approach to tumor antigen discovery and the development of novel proteomics reagents.” J Mol Biol 341(1): 161-9; Zhang, J., J. Tanha, et al. (2004). “Pentamerization of single-domain antibodies from phage libraries: a novel strategy for the rapid generation of high-avidity antibody reagents.” J Mol Biol 335(1): 49-56.)

FC5 sdAb Cloning, Expression and Purification

FC5 is a variable domain (V_{H}H) of the llama heavy chain antibody with encoding mRNA and amino acid sequences deposited in the GenBank (No. AF441486 and No. AAL58846, respectively). DNA encoding FC5 was cloned into the BbsI/BamHI sites of plasmid pSJF2 to generate expression vector for FC5. The DNA constructs were confirmed by nucleotide sequencing on 373A DNA Sequencer Stretch (PE Applied Biosystems) using primers fdTGIII, 5′-GTGAAAAAATTATTATTATTCGCAATTCCT-3′ and 96GIII, 5′-CCCTCATAGTTAGCGTAACG-3′. The FC5 was expressed in fusion with His₅ and c-myc tags to allow for purification by immobilized metal affinity chromatography using HiTrap Chelating™ column and for detection by immunochemistry, respectively. Single clones of recombinant antibody-expressing bacteria E coli strain TG1 were used to inoculate 100 ml of M9 medium containing 100 μg/ml of ampicillin, and the culture was shaken overnight at 200 rpm at 37° C. The grown cells (25 ml) were transferred into 1 L of M9 medium (0.2% glucose, 0.6% Na₂HPO₄, 0.3% KH₂PO₄, 0.1% NH4Cl, 0.05% NaCl, 1 mM MgCl₂, 0.1 mM CaCl₂) supplemented with 5 μg/ml of vitamin B1, 0.4% casamino acid, and 100 μg/ml of ampicillin. The cell culture was shaken at room temperature for 24 hours at 200 rpm and subsequently supplemented with 100 ml of 10× induction medium Terrific Broth containing 12% Tryptone, 24% yeast extract, and 4% glycerol. Protein expression was induced by adding isopropyl-β-D-thiogalactopyranoside (IPTG; 1 mM). After induction, the culture was shaken for an additional 72 hours at 25° C., and the periplasmic fraction was extracted by the osmotic shock method (Anand et al., 1991). The FC5 fragments were purified by immobilized metal-affinity chromatography using HiTrap Chelating column (Amersham Pharmacia Biotech; Piscataway, N.J.). FC5 produced was eluted in 10 mM HEPES buffer, 500 mM NaCl, pH 7.0, with a 10-500 mM imidazole gradient and peak fractions were extensively dialyzed against 10 mM HEPES buffer, 150 mM NaCl, 3.4 mM EDTA, pH 7.4. The molecular weight of FC5 is 13.2 kDa and that of FC5 fusion protein with c-myc and His₅ tags is 15.2 kDa.

FC5 was further engineered to add additional free cysteine that can be used for conjugation with drugs and carriers. DNA encoding sdAb FC5 was cloned into the BbsI/BamHI sites of plasmid pSJF2 to generate expression vector for monomeric FC5. cysFC5 gene was generated from FC5 template by a standard PCR using a forward primer that added a cysteine immediately after the His₅ ‘purification’ tag codons. cysFC5 gene was subsequently cloned into pSJF2 using standard cloning techniques. The integrity of the cloned construct was confirmed by nucleotide sequencing on 373A DNA Sequencer Stretch (PE Applied Biosystems, Streetsville, ON). cysFC5 was expressed in bacteria E coli strain TG1 and purified by immobilized metal-affinity chromatography (IMAC) as described previously [5,6]. The eluted fractions homogeneous for cysFC5 as judged by SDS-PAGE were pooled and extensively dialyzed against 10 mM HEPES buffer, 150 mM NaCl, 3.4 mM EDTA, pH 7.4. Protein concentrations were determined by the bicinchoninic acid assay (BCA).

To assure a complete reduction of the engineered free cysteine without compromising the conserved Cys22-Cys92 internal disulfide bonds, the cysFC5 was exposed to 50 mM Tris (2-Carboxyethyl)Phosphine Hydrochloride containing 5 mM EDTA in PBS overnight at 4° C. followed by rapid separation on G-25 sephadex columns prior to conjugation. These conditions did not compromise antigen binding activity of cysFC5 determined by intact cellular uptake and transmigration across CEC monolayers.

Procedure for Pentamerization of FC5 sdAb

Generation of pentameric single domain antibodies to improve the avidity of sdAbs was performed by fusing FC5 gene to the N-terminus of the D17E/W34A mutant of verotoxin B-subunit (VT1B). Briefly, VT1B gene was amplified by PCR using primers: forward: 5′-CCAGGGTTTTCCCAGTCACGAC-3′ and reverse: GCGGATAACAATTTCACACAGGAA. DNA encoding sdAb FC5 was cloned into BbsI/ApaI sites of plasmid pVT2 to generate expression vectors for pentavalent FC5. The obtained E. coli clone was designated P5. The DNA construct was confirmed by nucleotide sequencing. Pentameric protein P5 was produced by from E. coli cells by cell lysis. Briefly, the P5 clones were inoculated into 100 ml M9 medium supplemented with 0.4% casamino acids, 5 mg/l vitamin B1 and 200 μg/ml ampicillin and shaken overnight at 37° C. Thirty ml of the overnight M9 culture were transferred into 1 liter of M9 medium with the same supplements and shaken at 37° C. for 24 h. Induction of gene expression was initiated by the addition of 100 ml 10×TB nutrients, 200 μg/ml ampicillin and 1 mM IPTG and the cultures were shaken at room temperature for 72 h. For purification of the P5, the E. coli cell pellet of a 1-liter expression culture was resuspended in 25 ml of buffer containing 10 mM HEPES, 500 mM NaCl, 20 mM imidazole, and proteinase inhibitors cocktail tablet, pH 7.4). The cell suspension was lysed with an Emulsiflex Cell Disruptor (Avestin Inc. Ottawa, ON) and then incubated on ice with Dnase 1/1000 for 30 min. To clear the lysate, the protein sample was centrifuged for 10 min at 10,000 rpm at 4° C. followed by another spin 12,000 rpm for 40 min. The clear supernatant was loaded onto a Hi-Trap Chelating Affinity Column (Amersham Biosciences, Piscataway, N.J.) and purified by IMAC.

### Cell Culture

Primary human cerebromicrovascular endothelial cell (HCEC) cultures were isolated from human temporal cortex removed surgically from perifocal areas of brain affected by idiopathic epilepsy. Cells were dissociated, cultured and characterized as previously described in detail (Stanimirovic et al., 1996; Muruganandam et al., 1997). The morphological, phenotypic, biochemical and functional characteristics of these HCEC cultures have been described previously (Stanimirovic et al., 1996; Muruganandam et al., 1997). Passages 2-6 of HCEC were used for the experiments in this study.

Cell viability in the presence of FC5 and various pharmacological agents was assessed by the vital dye calcein-AM release assay as described previously (Wang et al., 1998).

The uptake of FC5 into HCEC was tested 15-90 minutes after adding 5 μg/ml of FC5 in the absence or presence of various pharmacological modulators of endocytosis. To visualize the intracellular distribution of FC5, cells were fixed, permeabilized and probed with the anti-c-myc antibody (1:100; 1 hour) followed by incubation with FITC-labeled anti-mouse IgG (1:250; 1 hour).

### In Vitro Blood-Brain Barrier Transport

Primary human CEC cultures were isolated, maintained and characterized. Passages 47 of different human CEC isolations were used for experiments in this study. Human CEC were seeded at a density of 80,000 cells onto 1 μm polycarbonate membrane filters dipped in 12-transwell plates. Transport studies were performed 7 days post seeding. Filter inserts were rinsed with transport buffer [PBS containing 5 mM glucose, 5 mM MgCl₂, 10 mM HEPES, 0.05% bovine serum albumin (BSA), pH 7.4] and allowed to equilibrate at 37° C. for 30 min. Experiments were initiated by adding 20 μg of sdAb or sdAb constructs to the apical chamber. Aliquots (100 μl) were collected from the basolateral chamber at various time intervals (5-120 min). 10 kDa- and 70 kDa-[¹⁴C] dextrans were used as molecular weight controls for mono- and pentameric FC5, respectively, and were quantified by liquid scintillation counting.

To measure the amount of FC5 or P5 in collected aliquots, 50 μl of aliquots were immobilized and dried overnight at room temperature in a nickel-NTA Hissorb 96-well plate (Nunc MaxiSorp). After blocking with 3% BSA in PBS for 2 h, anti-c-Myc monoclonal antibody tagged with HRP was added at a dilution of 1:5000 and detected with tetramethlybenzidine (TMB)/hydrogen peroxide (H₂O₂) substrate system (R&D Systems, Minneapolis, Minn.). To determine levels of IgG-HRP, aliquots were immobilized and dried overnight in a regular 96-well plate and quantified using TMB/H₂O₂ substrate system. The signal was measured at 450 nm on a microtiter plate reader. Unknown amounts of sdAb (monomer or pentamer) were determined from a standard curve constructed using known concentrations of respective sdAb protein.

### SDS-PAGE and Western Immunoblot

SDS-PAGE was carried out under non-reducing conditions with 15% polyacrylamide gels. For Western blotting, the separated proteins were electrophoretically transferred to a polyvinylidene fluoride (PVDF) membrane (Immobiolin P; Millipore, Nepean, ON). After blocking with 5% skim milk for 1 h, the membrane was probed for 2 h with anti c-Myc monoclonal antibody conjugated to peroxidase (dilution 1:5000); signal was detected by enhanced chemoluminescence.

### Laser-Capture Microdissection

Frozen brains from perfusion-killed animals were frozen-sectioned at 8-10 μm thickness on a cryostat (Jung CM3000, Leica, Germany). The sections were placed on Superfrost Plus microscope slides (Fisher Scientific, Nepean, ON) and kept at −80° C. until use. For LCM, the sections were warmed up at room temperature for 1 min, and brain vessels were stained by a rapid, 2-5 minute incubation with a fluorescently-tagged lectins Ulex Europeus Agglutinin I (UEA-I) (Vector Laboratories Inc, Burlington, ON) [1:20 dilution in dimethylpyrocarbonate (DEPC)-treated Milli-Q dH₂O], Griffonia Simplicifolia Lectin I-Isolectin B₄ (GSLI-Isolectin B₄) or Ricinus Communis Agglutinin I (RCA I) for human, mouse or rat brain, respectively. The sections were rapidly washed five times (3 sec/wash) in a phosphate buffer (0.2M phosphate buffer in DEPC water, pH 7.3) and then dehydrated by sequential exposures to the increasing concentrations of ethanol (70% ethanol for 30 sec, 96% ethanol for 30 sec, and 100% ethanol for 30 sec), followed by incubation in xylene for 5 min. The slides were air-dried for 5 to 10 min, and the fluorescent-labeled vessels were observed under the microscope.

LCM of vessels and vessel-free brain parenchyma was performed using a Pixcell II Laser Capture Microscope (Arcturus, Mountain View, Calif.). Laser spot size of 7.5-15 μm and a pulse power of 35-65 mW were applied. Approximately 15-20 captured microvessels were placed on one cap (CapSure LCM Caps, Arcturus, Mountain View, Calif.); 3 cups (50˜60 vessels) were collected from each section. The perivascular non-vessel containing parenchyma was also collected. During each step of LCM, images of tissues and microdissected cells were recorded. When LCM was completed, caps containing LCM-captured tissues were placed on a 0.5-ml Eppendorf tube (Brinkmann Instruments, Mississauga, ON) and stored at −80° C. until used for proteomics.

For ELISA, the film was removed from LCM caps, immobilized on a 96-well plate, blocked overnight with 3% BSA in PBS and then incubated with anti-c-Myc monoclonal antibody conjugated to HRP (1:5000) for 2 h. The bound antibody was detected using TMB/H₂O₂ substrate system.

### Immunohistochemistry

To study P5 binding to brain microvessels in situ, brain sections were first stained with vessel-selective fluorescein isothiocyanate (FITC)-labeled lectins. Sections were then incubated with P5 (1:100) for 1 h, washed, and blocked with 4% goat serum for 1 h. To detect P5, sections were first exposed to anti c-Myc monoclonal antibody (1:100) for 1 h followed by extensive washing, and then to Alexafluor 568-labeled anti-mouse secondary antibody (1:500) for 1 h. Imaging of the slides was performed using Zeiss LSM 410 (Carl Zeiss, Thornwood, N.Y.) inverted laser scanning microscope. Confocal images were obtained simultaneously to exclude artifacts from sequential acquisitions, using 488 and 568 nm excitation laser lines to detect FITC (BP505-550 emission), and Alexafluor 568 fluorescence (LP590 emission), respectively. For determining the fate of the P5 after crossing the blood brain barrier, co-localization of NeuN (neuronal marker) with the injected signal of P5 (tagged with Cy5.5). Brain sections were stained with monoclonal antibody against NeuN (abcam, 1:500) for 1 h and then detected using goat anti-mouse alexafluor 568 (invitrogen).

### Animal Optical Imaging

Mice were imaged by the time-domain small animal optical imaging system, eXplore Optix pre-clinical imager (GE Healthcare). Animals were either injected with the near-infrared fluorescent probe, Cy5.5 alone or FC5 (50 μg), P5 (250 μg) or NC11 (50 μg) labeled with Cy5.5 (all at similar equimolar concentration of 3 nM) via tail vein using a 0.5-ml insulin syringe with a 27-gauge fixed needle. Animals were then imaged in eXplore Optix 2, 6, or 24 h after drug injection. In all imaging experiments, a 670-nm pulsed laser diode with a repetition frequency of 80 MHz and a time resolution of 250 ps light pulse was used for excitation. The fluorescence emission at 700 nm was collected by a highly sensitive time-correlated single photon counting system and detected through a fast photomultiplier tube offset by 3 mm for diffuse optical topography reconstruction. Each animal was positioned prone on a plate that was then placed on a heated base (36° C.) in the imaging system. A two-dimensional mid-body scanning region encompassing the head was selected via a top-reviewing real-time digital camera. The optimal elevation of the animal was verified via a side viewing digital camera. The animal was then automatically moved into the imaging chamber for laser scanning. Laser excitation beam controlled by galvomirrors was then moved over the selected ROI. Laser power and counting time per pixel were optimized at 30 μW and 0.5 s, respectively. These values remained constant during the entire experiment. The raster scan interval was 1.5 mm and was held constant during the acquisition of each frame; 1024 such points were scanned for the region of interest (ROI). The data were recorded as temporal point-spread functions (TPSF) and the images were reconstructed as fluorescence intensity maps.

### Imaging Data Analysis

The background which consisted of baseline image before the antibody injection was subtracted (pixel by pixel) from each image. The time-domain fluorescence parameters were measured in every image of each animal using the same ROIs. The measured area was adjusted to approximately the same size for each animal. Ex-vivo whole brain or organ imaging analysis was also performed after saline perfusion to confirm the non-invasive imaging data.

To estimate the fluorescence parameters, eXplore Optix OptiView software program was used (Advanced Research Technologies, Montreal, QC). For reconstruction of in vivo imaging topography, 3D reconstruction software was used (Advanced Research Technologies, Montreal, QC). The topographic representations of the depth allowed visualization of Cy5.5 concentration and location within the animal profile.

### Proteomics

Trypsin Digestion

Each sample (pure antibodies or LCM-extracted samples) was precipitated by adding 10-volume of cold acetone and incubated at −20° C. for >12 h. Proteins were pelleted by centrifugation at 5000×g for 5 min and dissolved in 50 μL denaturing buffer (50 mM Tris-HCl, pH 8.5, 0.1% SDS, 4 mM DTT). Proteins were boiled for 15 min to denature and cooled for 2 min. To each sample, 5 μg of trypsin (Promega, cat # V5280) was added and samples were incubated at 37° C. for >12 h.

Purification on Cation Exchange (CE) Column

Each sample was diluted to 2 mL with CE load buffer (10 mM KH₂PO₄, pH 3.0, 25% acetonitrile) and pH was confirmed to be <3.3. Samples were purified on a cation exchange column (POROS® 50 HS, 50-μm particle size 4.0 mm×15 mm, Applied Biosystems, cat #4326695) as per manufacturer's protocol.

Mass Spectrometry and Database Searching

A hybrid quadrupole time-of-flight MS (Q-TOF™ Ultima, Waters, Millford, Mass., USA) with an electrospray ionization source (ESI) and an online reverse phase nanoflow liquid chromatography column (nanoLC, 0.3 mm×15 cm PepMap C18 capillary column, Dionex/LC-Packings, San Francisco, Calif., USA) was used for all analyses. The gradient of the nanoLC column used was 5-95% acetonitrile 0.2% formic acid in 90 min, 0.35 μL/min supplied by a CapLC HPLC pump (Waters).

Pure antibodies were first analyzed by nanoLC-MS and data-dependent nanoLC-MS/MS to identify all the ionizible peptides. LCM-extracted samples were then analyzed by nanoLC-MS in a survey (MS-only) mode to quantify the intensity of all the peptides present in each sample. From the nanoLC-MS raw data of each sample, peak intensities corresponding to the abundance of each peptide was extracted as described earlier (Haqqani et al, FASEB J. 2005 November; 19:1809-21). Peptides having the same “coordinates” (i.e, m/z and retention times) as that or FC5/P5 pure antibodies were determined using MatchRx software. These peptides were included in a “target list” and sequenced in a nanoLC-MS/MS mode. MS/MS spectra were obtained only on 2+, 3+, and 4+ ions. These were then submitted to PEAKS search engine (Bioinformatics Solutions Inc., Ontario, Canada) to search against a NCBI nonredundant, trypsin-digested (allowing 2 missed cleavage) mammalian database.

Absolute quantification of P5 in LCM samples

The amount of protein in each LCM-extracted parenchyma from P5-injected animals was first estimated using a large amount of LCM-extracted parenchyma samples (30,000 shots) from naïve animals. To do this, a dilution series of naïve-LCM sample was made (0 to 500 ng) and compared with P5-LCM sample using nanoLC-MS/MatchRx analysis. To estimate the amount of P5 levels in P5-LCM sample, a dilution series of pure P5 (0.01-100 ng) was made and compared with P5-LCM sample using nanoLC-MS/MatchRx analysis.

### PEGylation

Prior to PEGylation, the disulfide bond on the cysteine of cysFC5 was reduced with TCEP (10 mM) in the presence of 10 mM EDTA for 1 h under inert nitrogen gas. The reduced cysFC5 was then conjugated with bifunctional maleimide linked PEG (20 Kda) (Nektar Therapeutics, USA) at a ratio of 1:1000 under inert nitrogen gas overnight. The product was then purified by size exclusion chromatography, eluting with PBS-EDTA buffer under nitrogen on a G25 column to remove excess reagents and byproducts.

### Construction of FC5-Targeted Liposomal Doxorubicin

Two approached to link the antibody to the liposome were demonstrated. In the first approach, DOGS-NTA lipid was included in the liposome lipids and FC5 via its poly-histidine tag will spontaneously binds to the NTA chain in the DOGS-NTA lipid. The second approach using DSPE-PEG-mal where the cysFC5 (FC5 engineered to have an extra cysteine amino acid) is reduced using TCEP and then incubated with liposomes having in their composition DSPE-PEG-mal and in this case a thioether bond is formed between the sulfahydryl group in the cysteine and the maleimide group at the DSPE-PEG-mal lipid. Briefly, Liposomes composed of lecithin, cholesterol, mPEG-DSPE, DOGS-NTA at molar percent ratios of 62, 31, 1.9, 5, respectively, were used for the DOGS-NTA antibody linkage approach or Liposomes composed of lecithin, cholesterol, mPEG-DSPE, DSPE-PEG-mal at molar percent ratios of 64.5, 32, 2.58, 0.64, respectively, were used for the DSPE-PEG-mal antibody linkage approach Doxorubicin was remotely loaded into the liposomes using 250 mM ammonium sulfate. Dialysis of extraliposomal ammonium sulfate created a pH gradient allowing doxorubicin to be encapsulated at high concentration in the liposomes. Control of liposome size was achieved by extrusion using 100 nm size cut. FC5 was then bound to the liposomal doxorubicin using NTA-His linkage. Doxorubicin formulas composed of FC5-targeted liposomes, or non-targeted liposomes or free drug, all at equal dose of 8.9 mg/kg of doxorubicin, were injected intravenously in the tail vein of mice. 24 h after, mice were subjected to transcardial saline perfusion, organs were dissected and homogenized. Doxorubicin was extracted from tissue homogenates using extraction buffer (1/10 dilution): 100 μl of homogenate, 100 μl water, 50 μl of a 10% (v/v) triton x-100 (150 μl/1.35 ml), 750 μl of acidified isopropanol (0.75 N HCl), (12.1H HCl solution—1/16 dilution or 1.25 ml HCl/18.75 ml isopropanol), mixed vigorously, and incubated overnight at −25 degrees C. Next day homogenates were centrifuged at 13000 rpms for 20 min and supernatant of 200 μl was loaded into microplate; doxorubicin was quantified fluorometrically (excitation 470 nm and emission 590 nm). Absolute quantities of doxorubicin in the brain were determined from a brain standard curve composed of known amounts of doxorubicin spiked in the brain tissue.

TABLE 1
Quantification of the monomeric (FC5) and pentameric (P5)
form of FC5 delivered to the brain using LC-MS/MS.

| | Amount of | | | Amount of |
|---|---|---|---|---|
| | antibody in | Protein amount in | % of total | antibody in |
| Antibody | LCM sample | LCM sample | protein | the brain |
| FC5 | 0.378 ng | 135 ng | 0.28% | 90 ug |
| P5 | 0.789 ng | 176 ng | 0.45% | 144 ug |

1. A subunit comprising a polypeptide having an antibody or fragment thereof with affinity for an epitope found on the blood-brain barrier joined to a multimerization domain.

2. The subunit of claim 1 further including a cargo substance.

3. The subunit of claim 1 wherein the multimerization domain comprises a portion of a verotoxin B-subunit.

4. A method of enhancing the uptake of a cargo substance into a cell, said method comprising linking a targeting region and a multimerization domain to the cargo substance.

5. The method of claim 4 wherein the targeting region has an affinity for a blood-brain barrier antigen.

6. The method of claim 4 wherein the multimerization domain comprises a portion of a verotoxin B-subunit.

7. The method of claim 4 or 6 wherein the cell is a cancer cell.

8. The method of claim 4 wherein the cell is a mammalian cell other than a cancer cell.

9. (canceled)

10. The method of claim 23 wherein the cell is a polarized cell.

11. The method of claim 10 wherein the epitope is a blood-brain barrier epitope.

12. A kit comprising a plurality of subunits according to claim 2 and instructions for their administration to a patient.

13. (canceled)

14. The method of claim 21 wherein the cell type of interest is an endothelial cell.

15. The method of claim 21 wherein the cell type of interest is a cancer cell.

16. The method of claim 21 wherein the cell type of interest is a mammalian cell other than a cancer cell.

17. (canceled)

18. The method of claim 13 wherein the transport is receptor-mediated transcytosis.

19. (canceled)

20. A method of obtaining information useful in the diagnosis or treatment of a brain disease in a patient; said method comprising administering to the patient a plurality of subunits comprising a targeting region having affinity for a blood-brain barrier antigen, a multimerization domain, and a cargo substance including an agent.

21. A method of causing or enhancing transport of a compound by a cell type of interest, said method comprising:
obtaining a subunit comprising a multimerizing region and a targeting region, the targeting region having affinity for an epitope present on an accessible surface of the cell type of interest, and
allowing specific binding of one or more multimeric complexes on the cell type of interest.

22. The method of claim 21 wherein a cargo substance is functionally linked to the subunit.

23. The method of claim 21 wherein the receptor-mediated transcytosis.

24. The method of claim 23 wherein a cargo substance is functionally linked to the subunit.


SEQUENCE LISTING (52 sequences)

SEQ ID NO: 1 - protein, 122 aa, Artificial Sequence
  Description of Artificial Sequence Synthetic protein
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 2 - protein, 218 aa, Artificial Sequence
  Description of Artificial Sequence Synthetic protein
STPDCVTGKVEYTKYNDEDTFTVKVGDKELFTNRANLQSLLLSAQITGMTVTIKTNACHNGGGFSEVIFRGGGGSGLAGSEVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSEQKLISEEDLNHHHHH

SEQ ID NO: 3 - DNA, 832 bp, Artificial Sequence
  Description of Artificial Sequence Synthetic nucleotide construct
gttncgantgnntngagggtagaattcatgaaaaaaancgcnatcgcgatcnngttgccttgnctggtttcgctnccgntgcgcagnccgncttcgtacnatccgggcccggcaggcggcatccggtggcggcggttccacgcntgattgtgtaactggtaaggtggngtatacaaaatataatgatgaagatacctttacagttaaagtggnngataaagaattatttaccaacagagcgaatcttcannctcttcttctcagtgcgcnaattacggngatgactgtaaccattaaaactaatgcctgncataatgnagggggattcagcgaanttatttttcgtggctggaggtaggttccngagatgtgcagctgcaggcgtctggaggaggattggtgcaggctgggggctctctgagactctcctgtgcagcctctggattcaaaatcactcactataccatgggctggttccgccaggctcnagggaaggagcgtgaatttgtatcacgtattacttggggcggtgataacaccttctattcaaactccgtgaagggcccattcaccatttccagagacaacgcccagaacactttntatctncaaatgaacancctgnacctnaggacacggccgattnttactgtgcancacgntcgacgtncactgcgacnccncttagggtggactactggggcaaaggacccaggtcaccgtctcctcagganccaacaaanctgatccgcgangaanatctgactntcnccatcacnnttagtgaanctngnactggccgcgtttacaacgtcnngctggaaaccctncg

SEQ ID NO: 4 - DNA, 366 bp, Lama glama
gaggtccagctgcaggcgtctggaggaggattggtgcaggctgggggctctctgagactctcctgtgcagcctctggattcaaaatcactcactataccatgggctggttccgccaggctccagggaaggagcgtgaatttgtatcacgtattacttggggtggtgataacaccttctattcaaactccgtgaagggccgattcaccatttccagagacaacgccaagaacacggtctatctgcaaatgaacagcctgaaacctgaggacacggccgattattactgtgcagcaggttcgacgtcgactgcgacgccacttagggtggactactggggcaaagggacccaggtcaccgtctcctca

SEQ ID NO: 5 - protein, 21 aa, Artificial Sequence
  Description of Artificial Sequence Synthetic peptide
MKKTAIAIAVALAGFATVAQA

SEQ ID NO: 6 - protein, 5 aa, Artificial Sequence
  Description of Artificial Sequence Synthetic peptide
DVQLQ

SEQ ID NO: 7 - protein, 5 aa, Artificial Sequence
  Description of Artificial Sequence Synthetic peptide
GGGGS

SEQ ID NO: 8 - protein, 11 aa, Artificial Sequence
  Description of Artificial Sequence Synthetic peptide
EQKLISEEDLN

SEQ ID NO: 9 - protein, 5 aa, Artificial Sequence
  Description of Artificial Sequence Synthetic peptide
HHHHH

SEQ ID NO: 10 - protein, 12 aa, Artificial Sequence
  Description of Artificial Sequence Synthetic peptide
AAGSTSTATPLR

SEQ ID NO: 11 - protein, 11 aa, Artificial Sequence
  Description of Artificial Sequence Synthetic peptide
AGSTSTATPLR

SEQ ID NO: 12 - protein, 13 aa, Artificial Sequence
  Description of Artificial Sequence Synthetic peptide
CAAGSTSTATPLR

SEQ ID NO: 13 - protein, 10 aa, Artificial Sequence
  Description of Artificial Sequence Synthetic peptide
DNTFYSNSVK

SEQ ID NO: 14 - protein, 19 aa, Artificial Sequence
  Description of Artificial Sequence Synthetic peptide
DVQLQASGGGLVQAGGSLR

SEQ ID NO: 15 - protein, 10 aa, Artificial Sequence
  Description of Artificial Sequence Synthetic peptide
ITHYTMGWFR

SEQ ID NO: 16 - protein, 15 aa, Artificial Sequence
  Description of Artificial Sequence Synthetic peptide
ITWGGDNTFYSNSVK

SEQ ID NO: 17 - protein, 9 aa, Artificial Sequence
  Description of Artificial Sequence Synthetic peptide
LSCAASGFK

SEQ ID NO: 18 - protein, 25 aa, Artificial Sequence
  Description of Artificial Sequence Synthetic peptide
NSLKPEDTADYYCAAGSTSTATPLR

SEQ ID NO: 19 - protein, 32 aa, Artificial Sequence
  Description of Artificial Sequence Synthetic peptide
NTVYLQMNSLKPEDTADYYCAAGSTSTATPLR

SEQ ID NO: 20 - protein, 33 aa, Artificial Sequence
  Description of Artificial Sequence Synthetic peptide
NTVYLQMNSLKPEDTADYYCAAGSTSTATPLRV

SEQ ID NO: 21 - protein, 19 aa, Artificial Sequence
  Description of Artificial Sequence Synthetic peptide
SGFKITHYTMGWFRQAPGK

SEQ ID NO: 22 - protein, 13 aa, Artificial Sequence
  Description of Artificial Sequence Synthetic peptide
SGGGLVQAGGSLR

SEQ ID NO: 23 - protein, 24 aa, Artificial Sequence
  Description of Artificial Sequence Synthetic peptide
SLKPEDTADYYCAAGSTSTATPLR

SEQ ID NO: 24 - protein, 25 aa, Artificial Sequence
  Description of Artificial Sequence Synthetic peptide
SLKPEDTADYYCAAGSTSTATPLRV

SEQ ID NO: 25 - protein, 9 aa, Artificial Sequence
  Description of Artificial Sequence Synthetic peptide
THYTMGWFR

SEQ ID NO: 26 - protein, 6 aa, Artificial Sequence
  Description of Artificial Sequence Synthetic peptide
TMGWFR

SEQ ID NO: 27 - protein, 14 aa, Artificial Sequence
  Description of Artificial Sequence Synthetic peptide
YTMGWFRQAPGKER

SEQ ID NO: 28 - protein, 27 aa, Artificial Sequence
  Description of Artificial Sequence Synthetic peptide
AQAVFVRSGPGGGSGGGGSTPDCVTGK

SEQ ID NO: 29 - protein, 25 aa, Artificial Sequence
  Description of Artificial Sequence Synthetic peptide
AVFVRSGPGGGSGGGGSTPDCVTGK

SEQ ID NO: 30 - protein, 8 aa, Artificial Sequence
  Description of Artificial Sequence Synthetic peptide
DKELFTNR

SEQ ID NO: 31 - protein, 10 aa, Artificial Sequence
  Description of Artificial Sequence Synthetic peptide
EEDLNHHHHH

SEQ ID NO: 32 - protein, 9 aa, Artificial Sequence
  Description of Artificial Sequence Synthetic peptide
GGFSEVIFR

SEQ ID NO: 33 - protein, 12 aa, Artificial Sequence
  Description of Artificial Sequence Synthetic peptide
GGGSGLAGSEQK

SEQ ID NO: 34 - protein, 13 aa, Artificial Sequence
  Description of Artificial Sequence Synthetic peptide
LISEEDLNHHHHH

SEQ ID NO: 35 - protein, 15 aa, Artificial Sequence
  Description of Artificial Sequence Synthetic peptide
NACHNGGGFSEVIFR

SEQ ID NO: 36 - protein, 9 aa, Artificial Sequence
  Description of Artificial Sequence Synthetic peptide
NDEDTFTVK

SEQ ID NO: 37 - protein, 11 aa, Artificial Sequence
  Description of Artificial Sequence Synthetic peptide
NGGGFSEVIFR

SEQ ID NO: 38 - protein, 18 aa, Artificial Sequence
  Description of Artificial Sequence Synthetic peptide
PGGGSGGGGSTPDCVTGK

SEQ ID NO: 39 - protein, 26 aa, Artificial Sequence
  Description of Artificial Sequence Synthetic peptide
QAVFVRSGPGGGSGGGGSTPDCVTGK

SEQ ID NO: 40 - protein, 11 aa, Artificial Sequence
  Description of Artificial Sequence Synthetic peptide
SEEDLNHHHHH

SEQ ID NO: 41 - protein, 20 aa, Artificial Sequence
  Description of Artificial Sequence Synthetic peptide
SGPGGGSGGGGSTPDCVTGK

SEQ ID NO: 42 - protein, 16 aa, Artificial Sequence
  Description of Artificial Sequence Synthetic peptide
TNACHNGGGFSEVIFR

SEQ ID NO: 43 - protein, 15 aa, Artificial Sequence
  Description of Artificial Sequence Synthetic peptide
VEYTKYNDEDTFTVK

SEQ ID NO: 44 - protein, 10 aa, Artificial Sequence
  Description of Artificial Sequence Synthetic peptide
VGDKELFTNR

SEQ ID NO: 45 - protein, 10 aa, Artificial Sequence
  Description of Artificial Sequence Synthetic peptide
YNDEDTFTVK

SEQ ID NO: 46 - protein, 5 aa, Artificial Sequence
  Description of Artificial Sequence Synthetic peptide
KNLMG

SEQ ID NO: 47 - protein, 16 aa, Artificial Sequence
  Description of Artificial Sequence Synthetic peptide
TISGSGGTNYASSVEG

SEQ ID NO: 48 - protein, 4 aa, Artificial Sequence
  Description of Artificial Sequence Synthetic peptide
AFAI

SEQ ID NO: 49 - DNA, 30 bp, Artificial Sequence
  Description of Artificial Sequence Synthetic primer
gtgaaaaaattattattattcgcaattcct

SEQ ID NO: 50 - DNA, 20 bp, Artificial Sequence
  Description of Artificial Sequence Synthetic primer
ccctcatagttagcgtaacg

SEQ ID NO: 51 - DNA, 22 bp, Artificial Sequence
  Description of Artificial Sequence Synthetic primer
ccagggttttcccagtcacgac

SEQ ID NO: 52 - DNA, 24 bp, Artificial Sequence
  Description of Artificial Sequence Synthetic primer
gcggataacaatttcacacaggaa